(19) United States
(12) Patent Application Publication
(10) Pub. No.: US 2010/0248226 A1
(43) Pub. Date: Sep. 30, 2010

(54) METHODS FOR MEASURING CELL RESPONSE TO DNA DAMAGING AGENTS USING PROMYELOCYTIC LEUKEMIA PROTEIN NUCLEAR BODIES

(76) Inventors: Graham Dellaire, Halifax (CA); David Bazett-Jones, Toronto (CA)

Correspondence Address: KNOBBE MARTENS OLSON & BEAR LLP, 2040 MAIN STREET, FOURTEENTH FLOOR, IRVINE, CA, 92614, US

(21) Appl. No.: 12/376,432

(22) PCT Filed: Aug. 3, 2007
(86) PCT No.: PCT/CA07/01367

§ 371 Date: Feb. 4, 2009

(30) Foreign Application Priority Data
Aug. 4, 2006 (US) 60835438

(51) Int. Cl. C12Q 1/02; C12Q 1/68

(52) U.S. Cl. 435/6; 435/29

(57) ABSTRACT

Methods for assessing the potential efficacy of a DNA-damaging agent as a tumour treatment or for monitoring the efficacy of such treatment by determining promyelocytic leukemia protein nuclear body (PML NB) properties of the tumour are disclosed. PML NB properties, including morphology, number and biochemical composition, of cells or tissue are assessed following exposure to a DNA-damaging agent and compared to corresponding PML NB properties of cells or tissue not exposed to the DNA-damaging agent. The difference, or lack thereof, determines the efficacy of the DNA-damaging agent as a treatment for tumours or monitoring the efficacy of such treatment.

### FIELD OF THE INVENTION

The invention relates to methods for monitoring cellular responses to stress, including response to DNA damage-inducing agents. It further provides methods for monitoring the efficacy of anti-cancer treatments, either the potential efficacy before treatment or efficacy during treatment. The invention further provides methods for assessing oncogenesis in a subject.

### ABBREVIATIONS

The following abbreviations are used herein:

AT, Ataxia telangiectasia; ATLD, AT-like Syndrome; ATR, ataxia telangiectasia and Rad3-related; CHX, cycloheximide; DNA-PK, DNA protein kinase, Dox, doxycycline; NB, nuclear body; DSB, DNA double-strand breaks; ESI, electron spectroscopic imaging; Gy, Gray; GFP, green fluorescent protein; ICD, interchromatin domain; IR, ionising radiation; nuclear body; LM, light microscopy; MEF, murine embryonic fibroblasts; MRN, Mre11/Rad50/Nbs1; NBS, Nijmegan Breakage Syndrome; NHDF, normal human diploid fibroblasts; PML, promyelocytic leukemia; SSB, DNA single-strand breaks; SUMO-1, small ubiquitin-like modifier-1.

### BACKGROUND OF THE INVENTION

Mammalian nuclei contain 10 to 30 spherical structures called PML nuclear bodies (NBs) since a major component of these bodies is promyelocytic leukemia (PML) protein. The PML protein occurs in at least seven alternatively spliced isoforms and NBs in different tissues contain different isoform profiles (Jensen et al., 2001).

The PML protein and PML NBs are implicated in a number of cellular processes including transcriptional regulation, tumour suppression, apoptosis, DNA repair and the replication of both viral and cellular DNA (reviewed in Zhong et al., 2000; Everett 2001; Salomoni and Pandolfi, 2002; Dellaire and Bazett-Jones, 2004). How they contribute to these nuclear activities, however, has remained elusive. In normal mammalian cells, the PML protein co-accumulates in 5-30 NBs (Dellaire and Bazett-Jones, 2004) with as many as 75 other proteins (listed in the Nuclear Protein Database (NPD), Dellaire et al., 2003). Rather than just sequestering these proteins, there is compelling evidence that the bodies serve as sites for the post-translational modification of nuclear proteins. For example, the co-accumulation of p53, CBP and HIPK2 in PML NBs contributes to the regulated phosphorylation (by HIPK2) and acetylation (by CBP) of p53 in response to DNA damage (Hofmann et al., 2002; D'Orazi et al., 2002).

The structural and dynamic behaviour of PML NBs is intimately linked to the cell's chromatin integrity (Eskiw et al., 2004; Dellaire et al., 2006). Extensive chromatin contacts on the periphery of the protein cores of the NBs may account for their positional stability through extended periods in interphase of the cell cycle. Physical contacts with chromatin may be important for their proposed role in DNA replication. For example, early transcription and replication of the genomes of several DNA viruses occur immediately adjacent to PML NBs (reviewed in Everett 2001). A link between PML NBs and chromatin is also demonstrated in the maintenance of telomeres through a recombination mechanism called alternative lengthening of telomeres (ALT), whereby a subset of PML NBs in late S/G2 become associated with nascent DNA synthesis, DNA repair factors and telomere proteins (Yeager et al., 1999; Grobelny et al., 2000). The connection between PML NBs and chromatin also extends to a possible role for PML NBs in DNA repair mechanisms. For example, following DNA damage several DNA repair factors transit to and from PML NBs and the bodies themselves have been reported to co-localise with sites of unscheduled DNA synthesis in damaged cells (reviewed in Dellaire and Bazett-Jones, 2004). PML may also function in DNA damage signalling since PML null cells fail to fully activate p53 in response to DNA damage (Guo et al., 2000) and the PML protein is phosphorylated in response to DNA double-strand breaks (DSB) by Chk2 (Yang et al., 2002) and ATR kinase (Bernardi et al., 2004). It is unclear whether these modifications of PML or PML NB composition are critical for DNA repair to proceed or are a consequence of on-going repair. Regardless, PML NBs are clearly more than passive accumulations of nuclear proteins.

The dynamics and integrity of PML NBs appear to be closely linked to the state of chromatin in their vicinity. PML NBs exhibit radial symmetry and are positionally stable for the majority of interphase due to extensive chromatin contacts at their periphery (Eskiw et al., 2003; Dellaire et al., 2006). When the topological state of chromatin is altered during early S-phase by the replication of DNA, PML NBs lose both radial symmetry and integrity, fragmenting into “microbodies” by a fission mechanism (Dellaire et al., 2006). In addition, PML NB number can fluctuate during the cell cycle, increasing as cells progress through S-phase (Dellaire et al., 2006), and following DNA damage with ionising radiation (Carbone et al., 2002; Xu et al., 2003). Although topological changes in chromatin during DNA damage correlate with an increase in PML NB number, the mechanism responsible for the increase in PML NB number after DNA damage has not been elucidated.

Currently, cellular proliferation associated with a malignant cancer phenotype can be assayed by measuring Ki-67 nuclear antigen.

The cellular response to radiation or chemotherapy is measured by assaying either cell death (i.e. apoptosis) by microscopy using DNA end-labeling techniques (Gorczyca et al., 1993) and by gel electrophoresis analysis of internucleosomal DNA fragmentation (Comptom 1991), or by directly measuring the rejoining of DNA DSBs following treatment using techniques such as the Neutral Comet Assay (Olive et al., 1990). In addition, the formation of so-called “repair foci” containing either damaged chromatin containing the phosphorylated histone variant g-H2AX (Rogakou et al., 1999) or the accumulation of DNA repair factors such as the Mre11/Rad50/NBS1 (MRN) complex (Stracker et al., 2004).

For the analysis of tumour cell response to radiation or chemotherapy in patients, in addition to the analysis of apoptosis in tumour biopsies, the tumour volume is often assayed by MRI or PET scan weeks or months after treatment. Unfortunately, tumour volume as a marker for the efficacy of treatment is not ideal as the patient's cancer may advance further before it can be confirmed weeks or months later than the treatment is ineffective against their tumour.

There remains a need for improved methods for determining the cellular response to agents such as radiation and chemotherapeutic compounds which induce DNA damage.

### SUMMARY OF THE INVENTION

In one embodiment, the invention provides a method for assessing the potential efficacy of a DNA-damaging agent as a treatment for a tumor in a patient, the method comprising contacting a test portion of a sample of the tumor with the DNA-damaging agent; and

determining the value of a selected PML NB property in the test portion of the tumor sample at one or more time points after the contact with the DNA-damaging agent, wherein a difference in the value of the property in the test portion at one or more time points after the contact compared to the value of the property in a control sample indicates that the agent lacks efficacy for treatment of the tumor.

The control sample may be a portion of normal tissue from the biopsy sample or may be a sample of a normal tissue corresponding to the tumor.

The selected PML NB property may be NB number, NB biochemical composition, NB morphology or NB behaviour, such as motility, or a profile of NB number, composition, morphology or behaviour values at selected time intervals, or any PML NB characteristic which changes in response to DNA damage.

In a further embodiment, the invention provides a method for assessing the potential efficacy of a DNA-damaging agent as a treatment for a tumor in a patient, the method comprising contacting a test portion of a sample of the tumor with the DNA-damaging agent; and

determining PML NB numbers in the test portion of the tumor sample at one or more time points after the contact with the DNA-damaging agent, wherein a lack of increase in PML NB numbers in the tumor sample compared to the PML NB number of a control sample indicates that the agent lacks efficacy for the treatment of the tumor.

The control sample may be an untreated portion of the tumor sample or a portion of normal tissue from the biopsy sample.

In a further embodiment, the invention provides a method for monitoring the efficacy of a DNA-damaging agent administered to a patient to treat a tumor in the patient, the method comprising determining a selected PML NB property in a sample of the tumor obtained prior to administration of the agent and in at least one sample of the tumor obtained after administration of the agent, wherein a change in the PML NB property in the at least one post-administration tumor sample compared to the PML NB property of the pre-administration tumor sample indicates efficacy of the agent to treat the tumor.

Alternatively, the PML NB property determined in the tumor tissue may be compared with the value of that property in a corresponding normal tissue.

The selected PML NB property may be as described above.

The DNA-damaging agent may be radiation including but not limited to gamma-radiation, X-ray or other source of ionizing radiation, alpha-particle or Ultra Violet, or a chemotherapeutic compound including but not limited to reactive oxygen species (ROS) or chemicals that generate ROS (e.g. Hydrogen peroxide, Sodium Chromate, and Sodium arsenite), topoisomerase I (e.g. captothecin) and II inhibitors (e.g. VP16, doxorubicin), DNA alkylating/cross-linking agents (e.g. methyl methanesulfonate (MMS), N-Mthyl-N′-nitro-N-nitrosoguanidine (MNNG) cisplatin and Mitomycin C), Bioflavinoids (Genistein, Quercetin, Luteolin, Apigenin), or Radiomimetics (e.g. bleomycin, and neocarzinostatin).

In a further embodiment of this method, the PML NB property determined is PML NB number and an increase in PML NB number in the tumor tissue after treatment indicates likely efficacy of the treatment, whereas lack of an increase in PML NB number in the tumor tissue post-treatment suggests lack of efficacy.

In a further embodiment, the invention provides a method of monitoring the response of a cell or tissue to stress, the method comprising determining at least one PML NB property in the cell or tissue before and after the stress, or comparing the post-stress level of the property or properties to the level in a corresponding normal unstressed tissue. A change in the PML NB property or properties after stress may indicate the severity of the stress or the degree of damage to the cell or tissue due to the stress.

In a further embodiment, the invention provides a method for monitoring oncogenesis in a tissue in a subject, or for assessing the malignancy of a tumor, the method comprising determining at least one PML NB property in the tissue or tumor and comparing the level of the at least one PML NB property to that in a corresponding normal tissue. A change in the PML NB property in the patients tissue or in the tumor relative to that property in the corresponding normal tissue suggests increased proliferation in the tissue of the subject or increased malignancy in the tumor.

In one embodiment of this method, the PML NB property examined is PML NB number and the higher the number compared with normal tissue, the greater the likelihood of tumor development or progression.

In a further embodiment, the invention provides a method for assessing the potential efficacy of a DNA-damaging agent as a treatment for a tumor in a subject, the method comprising contacting a test portion of a sample of the tumor with the DNA-damaging agent;

determining the value of a selected promyelocytic leukemia nuclear body (PML NB) property in the test portion of the tumor sample at one or more time points after the contact with the DNA-damaging agent; and

comparing the one or more determined values of the selected property in the test portion of the tumor sample with the value of the selected property in a control portion of the tumor sample not contacted with the DNA-damaging agent,

wherein a lack of difference between the one or more determined values of the selected property in the test portion and the value of the property in the control portion indicates that the agent lacks efficacy for treatment of the tumor.

In a further embodiment, the invention provides a method for assessing the potential efficacy of a DNA-damaging agent as a treatment for a tumor in a subject, the method comprising contacting a portion of the tumor and a portion of normal tissue with the DNA-damaging agent;

determining the value of a selected PML NB property in the tumor and in the normal tissue at one or more time points after the contact with the DNA-damaging agent; and

comparing the one or more determined values of the selected property in the tumor and the normal tissue with the respective values of the selected property in a control portion of the tumor and a control portion of the normal tissue not contacted with the DNA-damaging agent to provide an indication of the response of the tumor and the normal tissue to the agent;

wherein a difference in response to the agent between the tumor and the normal tissue indicates that the agent lacks efficacy for treatment of the tumor.

In a further embodiment, the invention provides a method for assessing the potential efficacy of a DNA-damaging agent as a treatment for a tumor in a subject, the method comprising contacting a portion of the tumor and a portion of normal tissue with the DNA-damaging agent;

determining the value of a selected PML NB property in the tumor and in the normal tissue at several time points after the contact with the DNA-damaging agent; and

comparing the one or more determined values of the selected property in the tumor and the normal tissue with the respective values of the selected property in a control portion of the tumor and a control portion of the normal tissue not contacted with the DNA-damaging agent to provide an indication of the response of the tumor and the normal tissue to the agent;

wherein a difference in the timing of the response to the agent between the tumor and the normal tissue indicates that the agent lacks efficacy for treatment of the tumor.

In a further embodiment, the invention provides a method for monitoring the efficacy of a DNA-damaging agent administered to a subject to treat a tumor in the subject, the method comprising determining the value of a selected PML NB property in a sample of the tumor obtained prior to administration of the agent and in at least one sample of the tumor obtained after administration of the agent, wherein a change in the value of the PML NB property in the at least one post-administration tumor sample compared to the value of the PML NB property in the pre-administration tumor sample indicates efficacy of the agent to treat the tumor.

In a further embodiment, the invention provides a method for monitoring the efficacy of a DNA-damaging agent administered to a subject to treat a tumor in the subject, the method comprising determining the value of a selected PML NB property in at least one sample of the tumor and in a sample of normal tissue of the same type obtained after administration of the agent,

wherein a difference in the value of the PML NB property between the tumor sample and the normal tissue sample indicates lack of efficacy of the agent.

In a further embodiment, the invention provides a method for monitoring the efficacy of a DNA-damaging agent administered to a subject to treat a tumor in the subject, the method comprising determining the value of a selected PML NP property in the tumor and in the normal tissue at several time points after administration of the agent to provide an indication of the response of the tumor and the normal tissue to the agent;

wherein a difference in the timing of the response to the agent between the tumor and the normal tissue indicates that the agent lacks efficacy for treatment of the tumor.

In a further embodiment, the invention provides a method for monitoring the response of a cell or tissue to stress, the method comprising determining the level of at least one PML NB property in the cell or tissue before and after the stress, or comparing the post-stress level of at least one PML NB property in the cell or tissue to the level in a corresponding normal unstressed cell or tissue, wherein the greater the difference in the level of the at least one PML NB property in the stressed cell or tissue compared with the level in the cell or tissue before the stress, or with the level in a corresponding normal unstressed cell or tissue, the greater the response of the cell or tissue to the stress.

In a further embodiment, the invention provides a method for monitoring oncogenesis in a tissue in a subject, the method comprising determining the level of at least one PML NB property in the tissue, and comparing the level in the tissue to the level of the PML NB property in a normal control tissue, wherein a difference in the level of the property in the tissue of the subject relative to the level in the normal control tissue suggests increased proliferation and oncogenesis in the tissue of the subject.

In a further embodiment, the invention provides a method for assessing the malignancy of a tumor in a subject, the method comprising determining the level of at least one PML NB property in the tumor, and comparing the level in the tumor to the level of the PML NB property in a corresponding normal tissue, wherein the greater the difference in the level of the property in the tumor relative to the level in the corresponding normal tissue, the greater the likely malignancy of the tumor.

### SUMMARY OF THE DRAWINGS

Certain embodiments of the invention are described, reference being made to the accompanying drawings, wherein:

FIGS. 1A to 1F: PML NB number increases in response to DSBs in normal diploid human fibroblasts. NHDF cells (GM05757) were treated with varying doses of IR, etoposide (20 μM VP16) or 1.5 μM doxorubicin for 30 min to induce DSBs. Panel A) IF analysis of PML NB number in maximum intensity Z-projections of NHDFs following etoposide, time after treatment indicated in hours. Scale bars=5 μm. Panel B) IF analysis of the distribution of PML NBs in relation to DSBs in NDHFs after 2Gy IR or VP16. γ-H2AX is used as a marker for chromatin containing DSBs and white asterices mark the time points in which the maximum fluorescence intensity of γ-H2AX was first detected. White arrowheads indicate juxtaposition of γ-H2AX and PML NBs at 18 h post DNA damage (inset). Images represent a single focal plane. Scale bars=5 μm. Panel C) Comparison of mean PML NB number over time following IR, VP16 and doxorubicin treatment. Panel D) Comparison of fold increase in PML NB number over time following IR, VP16 and doxorubicin treatment. Panels E-F) Response of PML NBs to graded doses of IR expressed as a function of time (E) or at each time point as a function of dose (F).

FIGS. 2A to 2D: PML protein and PML NB dynamics following DSB induction. Panel A) PML microbody formation occurs rapidly after treatment with etoposide. Two U-2 OS human osteosarcoma cells stably expressing GFP-PML IV protein were imaged by fluorescence microscopy before (T=0) and after addition of etoposide (20 μM VP16) (T=5 min and T=2 h). Enlarged region of the cell marked by white asterix is shown at each time point. White arrowheads indicate newly formed microbodies following VP16 treatment. Panel B) Formation of PML microbodies in response to DNA double-strand breaks occurs by supramolecular fission from pre-existing parental PML NBs. A U-2 OS cell expressing GFP-PML IV was visualised before (T=0) and during treatment with 20 μM VP16 over several minutes (T=0.5, 1.0 and 1.5 min are shown). White arrowhead indicates fission of a PML microbody from a larger parental PML NB. Panel C) PML NBs increase in number in cells irradiated on ice. NHDFs (GM05757s) were incubated on ice for 20 min and either fixed (Control, white bar) or irradiated on ice (10 Gy IR, black bar) before fixation. Mean PML NB number increases significantly between Control (17±1, n=30) and cells irradiated with 10 Gy IR on ice (24±2, n=30)(*p=0.0008). Panel D) Dynamics of the PML protein within PML NBs is affected by DNA damage and reduced temperature. Asynchronous U-2 OS GFP-PML IV cells were subjected to treatment with etoposide (20 μM VP16, 30 min) before mobility of PML protein within PML NBs was analysed by FRAP at 37° C. (n=20, closed squares). Mobility of the PML protein at PML NBs in DNA damaged cells is compared to control untreated cells (n=20, open circles) at 37° C. and at 15° C. (n=7, open diamonds). Data presented as the mean fluorescence recovery plotted as percent of initial fluorescence intensity of the PML NB over 14 min. Error bars=s.e. and scale bars=5 μm.

FIGS. 3A to 3B: PML NBs lose positional stability when chromatin is damaged in their vicinity.

Panel A) UV-laser induced DSBs alter the positional stability of PML NBs. A single U-2 OS cell expressing GFP-PML IV is shown in which DSBs were created in a laser track along a defined region of interest (˜0.5×10 μm, white rectangle) by photo-induction; PML NB movement was tracked over time. PML NBs (white arrowheads) along the laser path (white rectangle) move towards and aggregate with one large PML NB (white arrow) adjacent to laser track. PML NB number (NB#) is shown before laser-induction of DSBs and 22 min post-induction. Panel B) Confirmation of laser-induced DSBs by IF detection of γ-H2AX. The same cell shown in A was fixed at 60 min post laser-induction of DSBs and processed for immunodetection of PML and γ-H2AX. PML NB number at this time point is indicated (NB#).

FIGS. 4A to 4C: Ultrastructural analysis of PML NBs in NHDFs by correlative LM/ESI before and after etoposide-induced DNA damage. Panel A) LM/ESI of a single NHDF (GM05757) cell, fluorescently labeled for PML protein. Elemental maps of nitrogen (N) and phosphorus (P), and the merged maps of a PML NB and its surrounding nucleoplasm reveal protein-based (cyan) and nucleic acid-based (yellow) components. Chromatin appears yellow in the merged image due to high N and P content. A single PML NB is shown at higher magnification (cyan, as indicated by the white arrow) making many contacts to the surrounding chromatin (yellow) and has radial symmetry typical of PML NBs in unstressed cells. Panel B) LM/ESI of a single NHDF (GM05757) treated with 20 μM etoposide (VP16) for 30 min, fluorescently labeled for PML protein. Following treatment with VP16, the protein core of PML NB is disrupted in response to DSB induction; few contacts with chromatin remain and radial symmetry is lost. Panel C) PML NB in B at higher magnification (left) and a cartoon representation of the same EM micrograph (right), where PML protein-containing protein structures (red), chromatin (yellow) and other non-chromosomal protein (blue) are shown. Redistribution of PML microbodies along chromatin fibres (white asterices) is observed and larger interchromatin spaces (black areas) are apparent. PML protein localization was determined by immunogold detection of PML (white dots). Scale bars=500 nm.

FIGS. 5A to 5C: The increase in PML NB number in response to DSBs is independent of new protein translation and p53. NDHF cells (GM05757) in the presence or absence of 150 μM cycloheximide (CHX), Saos-2 human osteosarcoma cells, and isogenic HCT116 human colon carcinoma cells (+ or −p53), were treated with etoposide (20 μM VP16) for 30 min. (*p<0.0001). Panel A) Western analysis of PML protein levels following etoposide treatment in presence or absence of cycloheximide. NHDFs were treated with etoposide (20 μM VP16 for 30 min) and harvested at the indicated times for SDS-PAGE and Western analysis. Ratio of PML protein levels in the control lane to PML protein at the indicated time points post-etoposide treatment are shown normalised against actin. Panel B) Comparison of mean PML NB number following VP16 treatment in NDHFs, NDHFs treated with cycloheximide (+CHX) and Saos-2 cells. Panel C) Comparison of mean PML NB number following VP16 treatment in isogenic HCT116 and HCT116 p53 null cells. Panel D) Comparison of DNA synthesis activity of NHDFs, NHDFs treated with cycloheximide (+CHX) and Saos-2 cells at 18 h post VP16 treatment. 18 h after VP16 treatment, cells were incubated with BrdU, fixed, processed for immunodetection of BrdU and DNA was counterstained with DAPI. White asterices=BrdU positive cells.

FIGS. 6A to 6B: The increase in PML NB number in response to DSBs is delayed or inhibited in the presence of P13 kinase inhibitors and in DNA repair-deficient cell lines.

Panel A) Comparison of effects of DNA repair kinase inhibitors on the increase in PML NB number in response to DSBs. NHDF cell line GM05757 (Control) was pre-treated with Chk2 kinase inhibitor (10 μM, Chk2 inhibitor II) or various PI3 kinase inhibitors (5 mM caffeine; 20 μM wortmannin, LY2942002, 50 μM) for 30 min prior to treatment with etoposide (20 μM VP16, 30 min)(*p<0.0001; **p<0.001). Panel B) Comparison of the fold increase in PML NB number following etoposide treatment (20 μM VP16, 30 min) in NHDF cells and DNA repair deficient human fibroblast cell lines (AT, ataxia telangiectasia; NBS, Nijmegan breakage syndrome; ATLD, AT-like disorder; Seckel, Seckel syndrome) (*p<0.0001; **p<0.02).

FIGS. 7A to 7C: PML NB induction in response to DSBs requires NBS1, Chk2 and ATR-function. Cells were treated with etoposide (20 μM VP16, 30 min), left to recover for 3 h and processed for IF detection of PML. DNA was counterstained with DAPI. PML NB number is indicated in maximum intensity Z-projections of IF images of control and etoposide treated cells (left) and a comparison of mean PML NB number (right) is shown. Scale bars=5 μm. Error bars=s.e.m. Panel A) Comparison of the PML NB number between etoposide treated Chk2 null (Chk2−/−) and wild type Chk2 (Chk2 WT) murine embryonic fibroblasts (*p<0.001). Panel B) Comparison of PML NB number between Tert-immortalised human NBS fibroblasts infected with an empty retroviral vector (NBST pBabe) or a retroviral vector carrying wild type human NBS1 (*p<0.02; **p<0.02). Panel C) Disruption of ATR kinase function inhibits the increase in PML NB number in response to DSBs. U-2 OS cells expressing a doxycycline-inducible kinase inactive dominant negative ATR kinase (ATR-DN) were treated with (+Dox) or without doxycycline for 24 h prior to etoposide treatment. Besides PML, IF detection of ATR-DN (Fl-ATR-DN) is also shown. Cells with high ATR-DN expression (white arrow) contain fewer PML NBs following etoposide treatment than cells with low expression (white arrowhead). White asterices indicate two cells with similar PML NB number in cells not expressing ATR-DN. DNA was counterstained with DAPI. (*p<0.0001; **p<0.001).

FIGS. 8A to 8C: Summary of the biophysical and molecular mechanisms responsible for the increase in PML NB number in response to DSBs.

Panel A) Model of the biophysical effect on PML NBs by changes in chromatin structure or tensegrity. Chromatin is constrained and under tension (double-headed arrows) by tethering to subnuclear compartments such as the nucleolus, nuclear lamina and possibly PML NBs. DSB-induced changes in chromatin structure or tensegrity alter the balance of forces constraining chromatin within the nucleus; this biophysical phenomenon destabilizes PML NBs that are tethered to chromatin, resulting in microbody formation by fission from pre-existing NBs. Panel B) Model for PML NB number increase in response to DSBs. Initially PML NB number increases due to biophysical changes in chromatin following DSBs as in A. The second phase of the PML NB response to DNA damage requires on-going DNA repair processes which can be inhibited by low temperatures (i.e. 4° C.) by inhibition (caffeine) or loss of ATR kinase function (AATR) and to a lesser extent by loss of function of NBS1 (ΔNBS1) or Chk2 (ΔChk2), whose activation is affected by inhibition of ATM (wortmannin). Panel C) Summary of DNA repair kinase pathways implicated in phosphorylation of the PML protein in response to DNA double-strand breaks. It is currently unknown if ATM can directly phosphorylate PML.

FIGS. 9A to 9C: Co-localisation of PML with γ-H2AX, NBS1 and RPA following the induction of DNA DSBs. NHDFs (GM05757) were treated with etoposide (20 μM VP16 for 30 min) and left to recover for the indicated times before being processed for immunofluorescence microscopy. DNA has been counterstained with DAPI. Line scans were carried out between the white arrowheads as indicated (from left to right) on each RGB image. Scale bars=5 μm. Panel A) PML co-localises with NBS1 and γ-H2AX at late time points following etoposide treatment. The localisation of PML (blue), NBS1 (green), and γ-H2AX (red) within cells is shown at the left and the relative levels of these factors are depicted on the right by line scans. White asterices adjacent to arrows indicate the position of PML NBs containing both NBS1 and γ-H2AX, which are also indicated on the line scans by black asterices. Panel B) PML co-localises with RPA following etoposide treatment in the sub-population of cells positive for RPA foci. The localisation of PML (green), and RPA (red) within cells is shown at the left and the relative levels of these factors are depicted on the right by line scans. Areas containing PML NBs that are juxtaposed to or partially co-localise with RPA foci are bound by a white box and are shown at higher magnification as an inset. Panel C) RPA foci following etoposide treatment appear in cells in S-phase and G2 of the cell cycle. NHDFs treated with etoposide were left to recover for 3 h before being processed for immunodetection of PML (green), RPA (red) and cell cycle markers for S-phase (Cyclin A) or G2 (serine 10 phosphorylated histone H3; pH3). Scale bars=5 μm. S-phase or G2 cells positive for cyclin A or pH3 (respectively) are indicated by white asterices.

FIGS. 10A and 10B: Modeling of PML NB number and characterisation of DSB-induction following etoposide treatment. Panel A) Mathematical modeling of the increase in PML NB number following IR. The increase in PML NB number following irradiation with graded doses of IR can be modeled using the equation y=a*b^{X}+c; where at 30 min, 3 h and 6 h a=−16, −11, −7, b=0.50, 0.364, 0.573, and c=33, 28, 24, respectively. Error bars=s.e.m. Panel B) Comparison of DSB induction by ionising radiation (IR) versus etoposide treatment. Neutral comet assay of NHDFs (GM05757) treated with either 2 and 5 Gy IR or etoposide (20 μM VP16 for 30 min). Olive Tail moment=the ratio of fragmented DNA contained within the DNA electro-eluted from the nucleus (Comet's tail) divided by the non-fragmented DNA within the nucleus (Comet's head).

FIGS. 11A to 11E: Relative levels of PML, SP100 and SUMO-1 following the induction of DNA DSBs. NHDFs (GM05757) were treated with etoposide (20 μM VP16 for 30 min) and left to recover for the indicated times before being processed for the immunodetection of the PML protein (green), SP100 or SUMO-1 (red). DNA has been counterstained with DAPI. Line scans were carried out between the white arrowheads as indicated (from left to right) on each RGB image. Scale bars=5 μm. Panel A) Comparison of PML and SP100 levels within PML NBs following etoposide treatment. The relative levels of PML versus SP100 are shown as line scans at the right. Panel B) Comparison of PML and SUMO-1 levels within PML NBs following etoposide treatment. The relative levels of PML versus SUMO-1 are shown as line scans at the right. Panel C) Quantification of PML and SUMO-1 line scans. The fluorescence intensity of PML or SUMO-1 signal in PML NBs above nucleoplasmic background was compared for untreated NHDFs (GM05757) versus cells treated with etoposide at different time points as indicated. The mean ratio of the fluorescence intensity of PML versus SUMO-1 signal is shown for each time point (n=22 scan measurements from an average of 5 cells)(*p<0.0001, **p<0.0002). Error bars=s.e. Panel D) SUMO-1 over-expression reduces PML NB number in NHDFs. NHDFs (GM05757) were transfected with GFP-SUMO-1, fixed and processed for the immunodetection of PML and SUMO-1. The mean number of PML NBs per cell was determined for untransfected control cells (Control, n=30) and compared to that of cells expressing either low (Low SUMO-1, n=20) or high levels of SUMO-1 (High SUMO-1, n=20). The levels of SUMO-1 were arbitrarily determined by first determining the average whole cell integrated fluorescence intensity of GFP-SUMO-1 signal, after which cells with fluorescence intensities below (low expressors) or above (high expressors) the average were binned together for analysis. (*p<0.01, **p<0.0001) Error bars=s.e. Panel E) Over-expression of SUMO-1 can delay or inhibit PML NB induction following etoposide treatment. NHDFs (GM05757) were transfected with GFP-SUMO-1 and 18 h post-transfection were treated with etoposide (20 μM VP16 for 30 min) and left to recovery for the indicated times before being fixed and processed for the immunofluorescence detection of GFP-SUMO-1 and PML. As in C, the PML NB number of cells was arbitrarily binned into two groups, those expressing a high level of SUMO-1 (High SUMO-1, n=15) and those with a low expression level (Low SUMO-1, n=15) for each time point post-etoposide treatment. The mean PML NB number is shown. Error bars=s.e.

FIGS. 12A to 12C: Characterisation of etoposide and caffeine treated normal human diploid fibroblasts (GM05757). Panel A) Comparison of the cell cycle profiles of NHDFs after etoposide treatment in the presence or absence of caffeine. NHDF (GM05757) cells were either treated with 5 mM caffeine (+Caf) for 30 min prior to, or left untreated (Control), before being exposed to etoposide (20 μM VP16 for 30 min). The cells were then left to recover for the indicated times in the presence (+Caf) or absence of 5 mM caffeine for the indicated time, before being processed for FACS analysis. The percentage of cells in G1, S and G2 phase of the cell cycle are shown. Panel B) Comparison of the PML NB induction profile of NHDF (GM05757) cells either treated with 5 mM caffeine for 30 min (black bars, 5 Gy+Caffeine) prior to, or left untreated (grey bars, 5 Gy), before being irradiated with 5 Gray of IR (*p<0.001). Panel C) Cell cycle profiles of the NHDFs treated with or without caffeine. NHDF cell line GM05757 was treated with or without 5 mM caffeine (Control) for the indicated time points before being harvested for fluorescent activated cell sorting (FACS) analysis. The percentage of cells in G1, S and G2 phase of the cell cycle are shown.

### DETAILED DESCRIPTION OF THE INVENTION

The inventors have recognized that PML nuclear bodies (NBs) are highly sensitive DNA damage sensors and that the number, composition, morphology and/or behaviour of PML nuclear bodies in a cell is reflective of the topological state of the cell's chromatin and the integrity of the DNA repair pathways involved in maintaining the integrity of the cell's DNA.

Determinations of these parameters of the PML nuclear bodies therefore provide methods for assessing many facets of a cell's DNA metabolism.

In one embodiment, the invention provides a method for assessing the potential efficacy of a cancer treatment such as radiation or chemotherapy before subjecting a patient to that treatment. In this embodiment, a biopsy sample of the patient's tumor which is to be treated is obtained by routine techniques, a portion of the sample is retained as a pre-treatment control and the rest of the sample is subjected to the proposed treatment in vitro, by contacting the sample with the selected chemotherapy reagent or by irradiation by methods known to those skilled in the art of in vitro analysis of DNA damaging agents. Alternatively, a portion of normal tissue obtained from the biopsy sample is used as control and is subjected to the proposed treatment along with the tumor sample.

The control sample and one or more portions of the treated biopsy sample are then examined for PML NB numbers by fixation of the samples, for example by paraformaldehyde, followed by processing for immuno-histochemical detection of the PML protein and visualization of NBs, for example by epifluorescence microscopy, as described in the examples herein. Portions of the treated biopsy sample are fixed and examined at various times after treatment, for example immediately following treatment and at time points from 30 mins to 48 hours after treatment, and the number of NBs is counted, as described in Example 1.

The profile of NB numbers in tumor cells can be compared with that in normal tissue contained in the biopsy sample and/or with the NB numbers in the pre-treatment control. If PML NB numbers in the tumor cells do not increase relative to the pre-treatment tumor sample, or if the response to the treatment seen in the normal tissue of the biopsy sample is not seen in the tumor sample, this indicates that the tumor is not responsive to the attempted treatment method.

Timing of the post-treatment examination of samples may be varied based on the known typical tissue response to the particular treatment or therapeutic drug.

It is also possible to determine a typical NB number response profile of a particular type of tissue, eg. breast tissue, to a particular DNA-damaging treatment by examining a representative number of samples. The NB number response profile of a tumor of that tissue, eg. a breast tumor, determined as described herein, may then be compared to the typical profile for the corresponding normal tissue. A change from the normal tissue NB number profile, such as a delayed peak response, seen in the tumor tissue may indicate that the tumor is unlikely to respond to that treatment.

In a second embodiment, the control sample and one or more portions of the treated biopsy sample are then examined for PML NB biochemical composition with respect to but not limited to SUMO-1 protein by immuno-histochemical detection of the PML protein and visualization of bodies by epifluorescence microscopy immediately following treatment and/or at time points between 30 min and 48 hours following treatment.

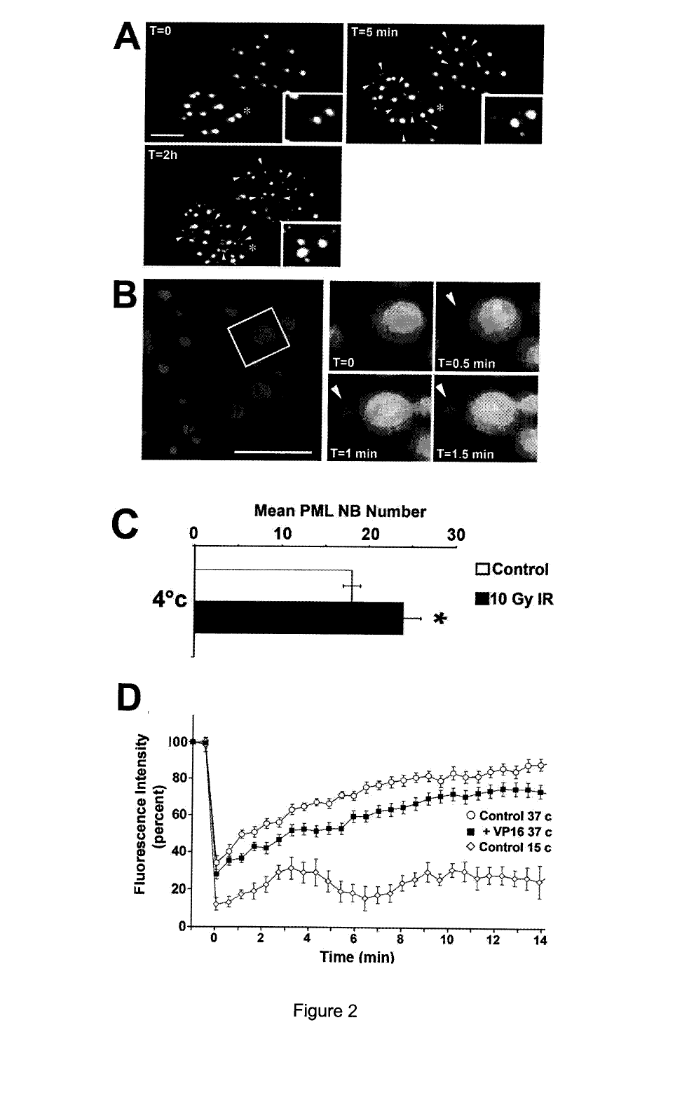

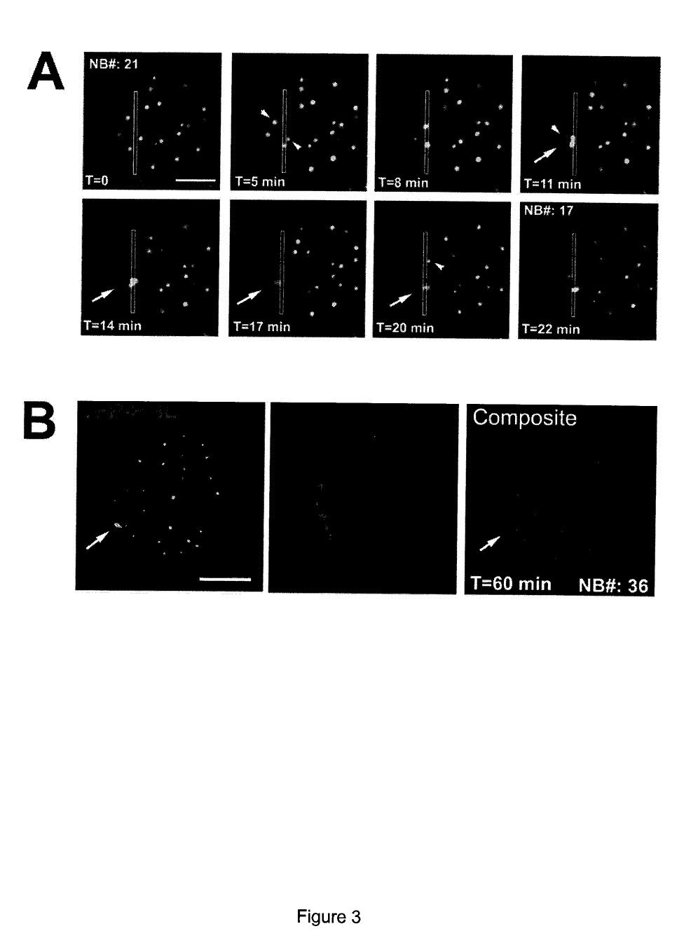

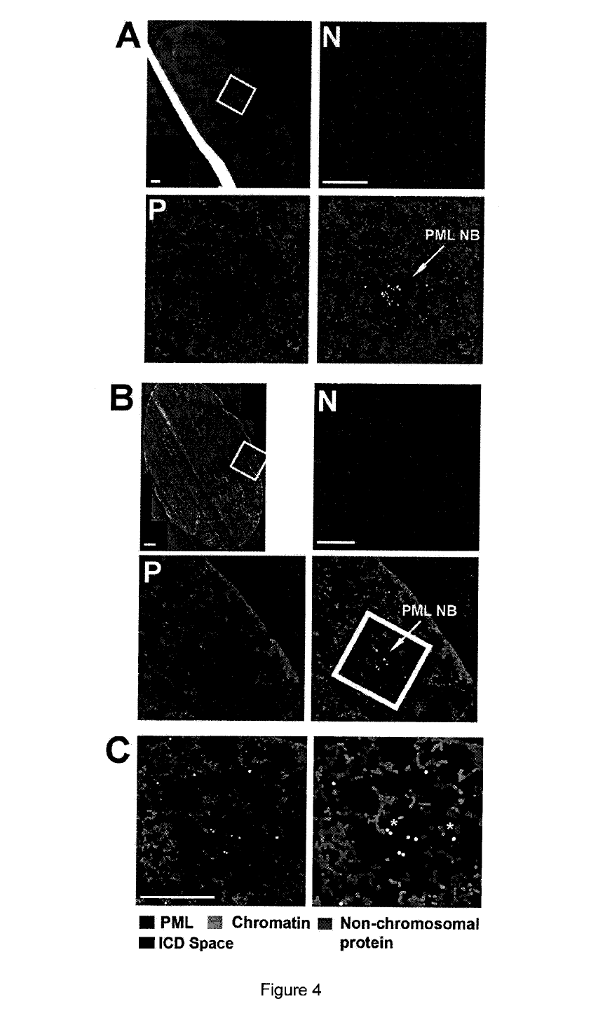

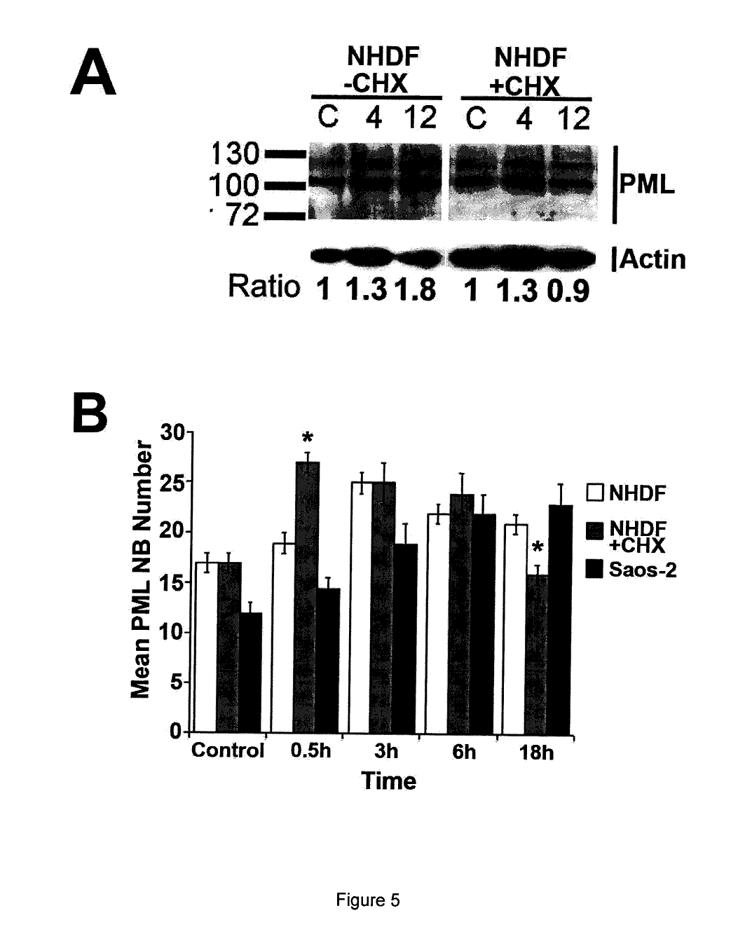

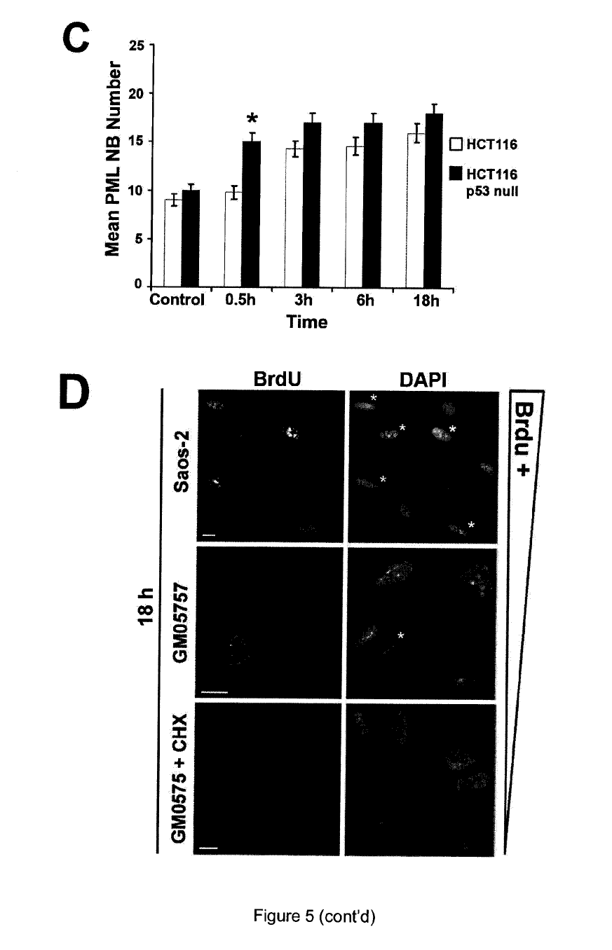

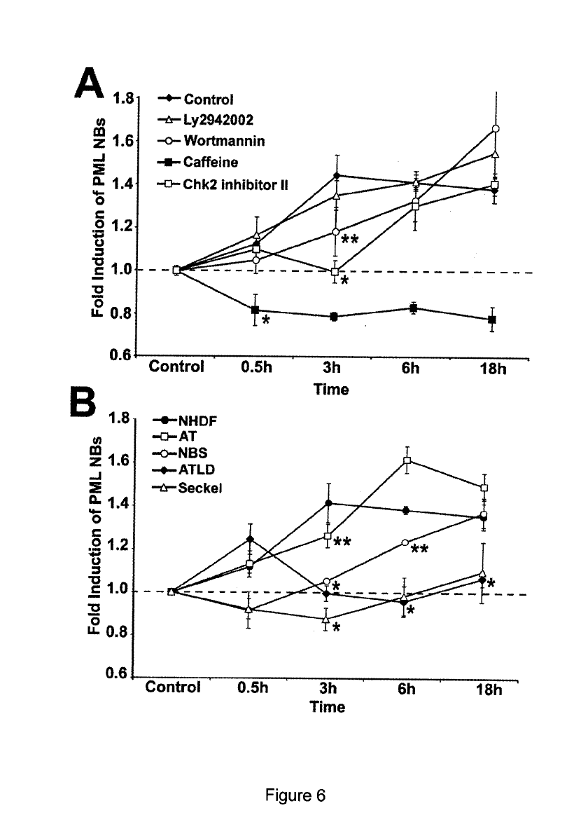

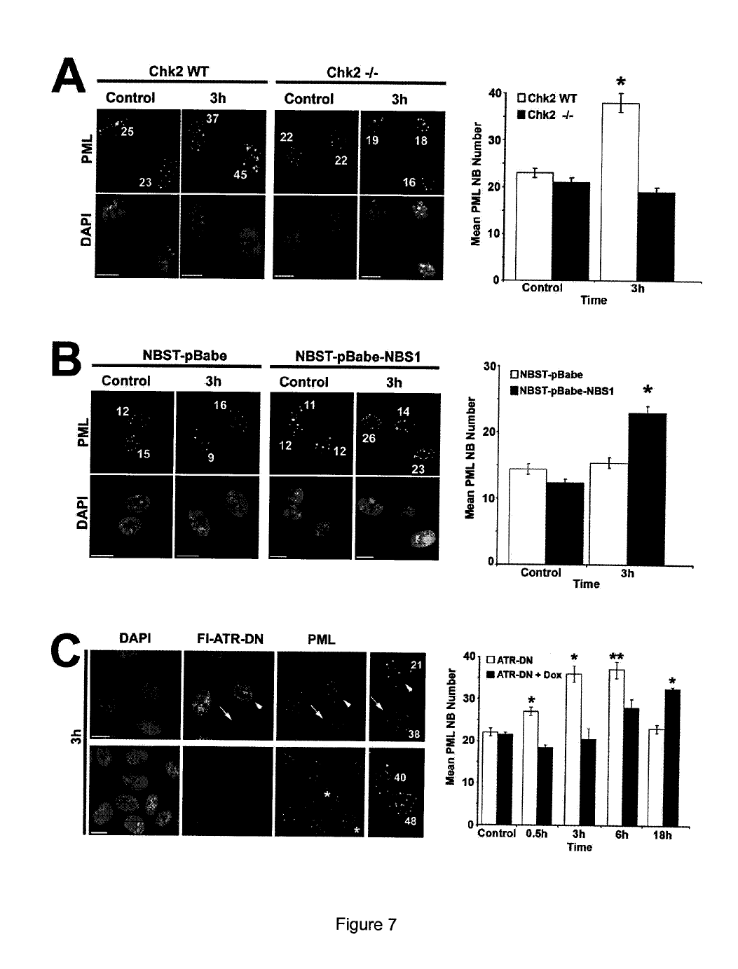

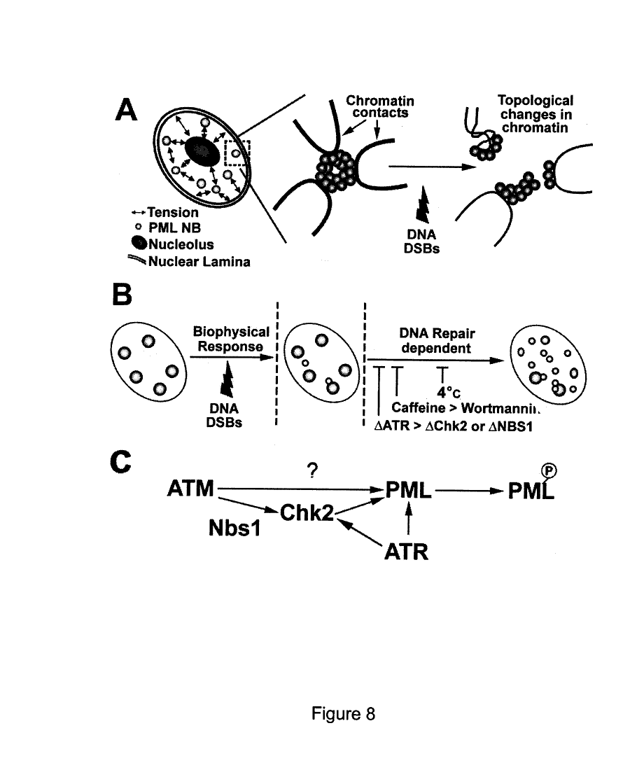

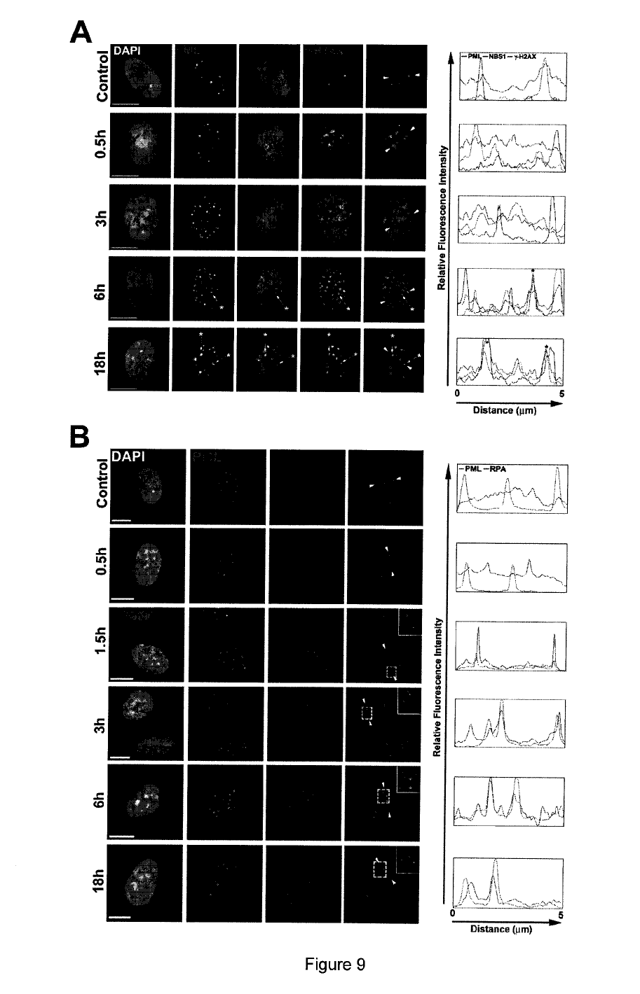

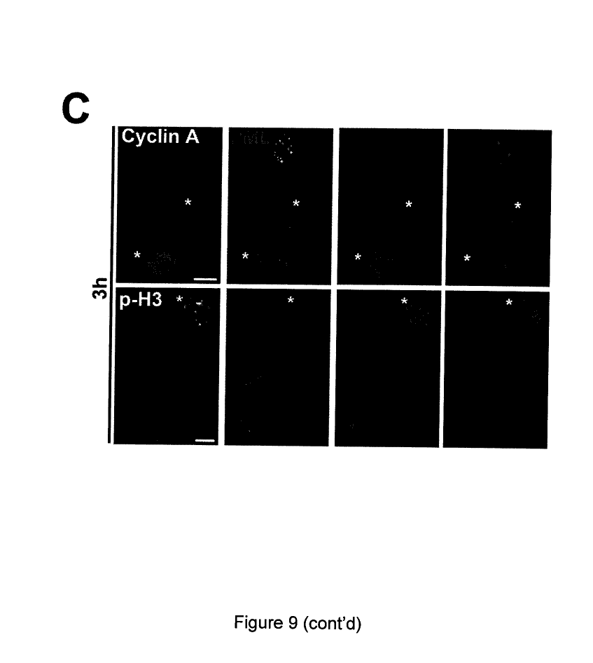

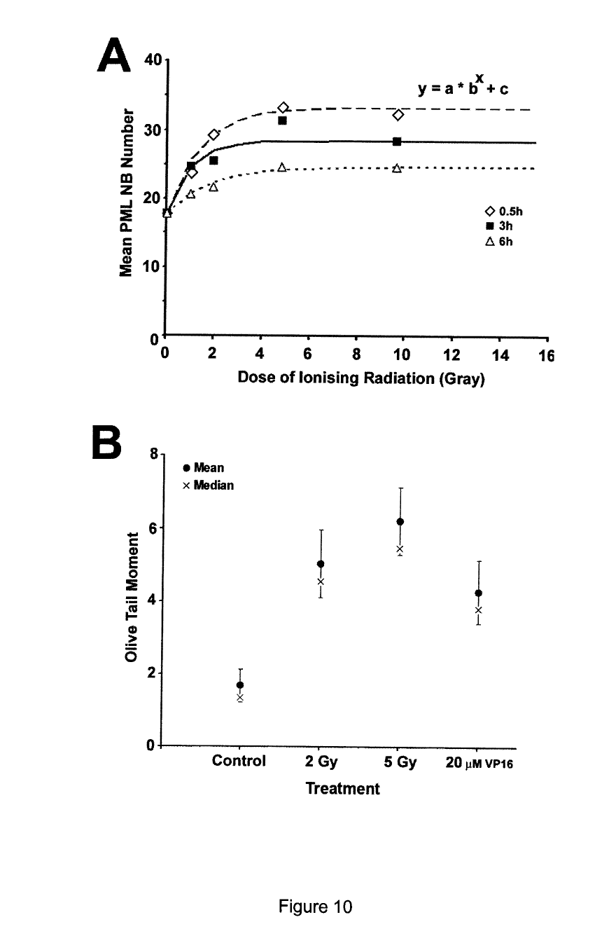

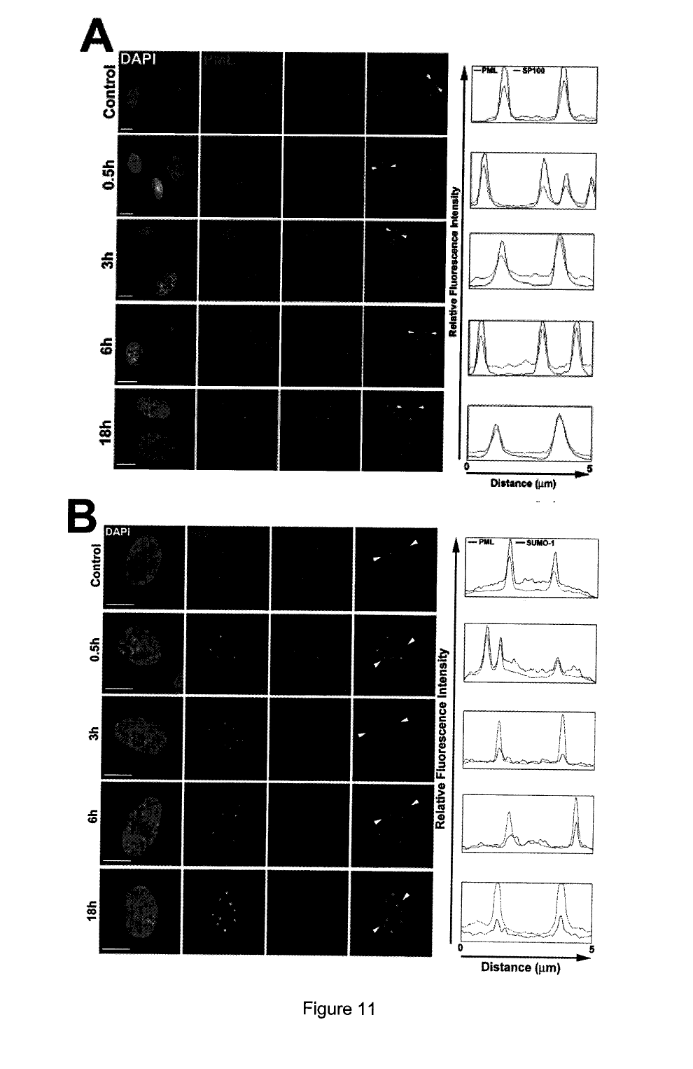

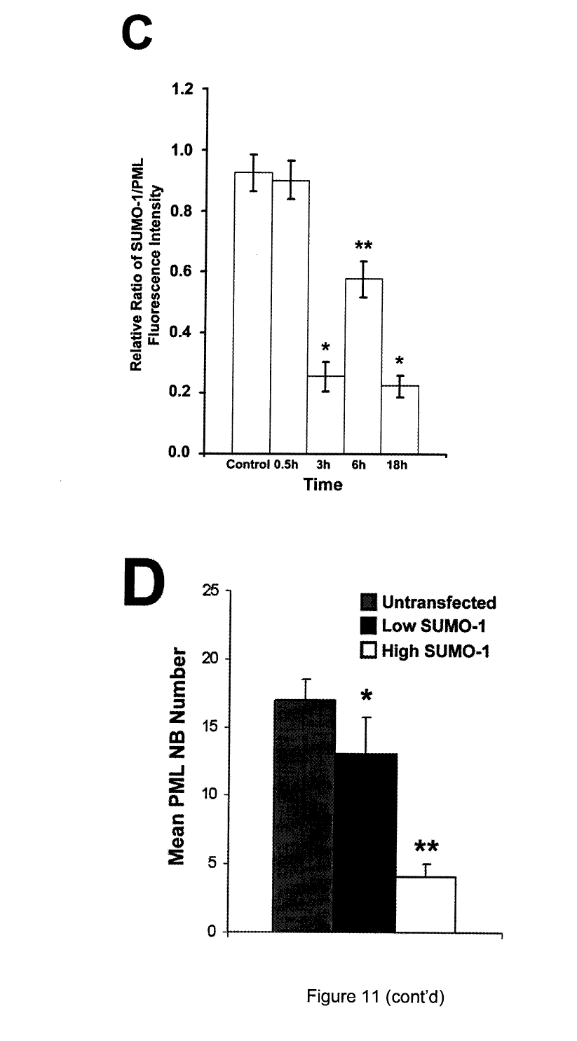

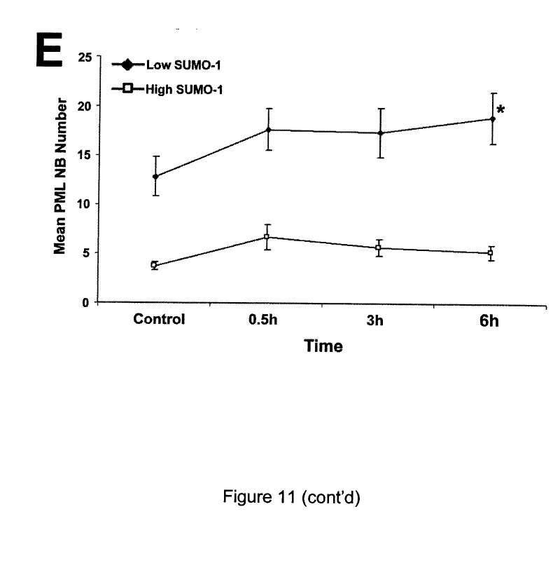

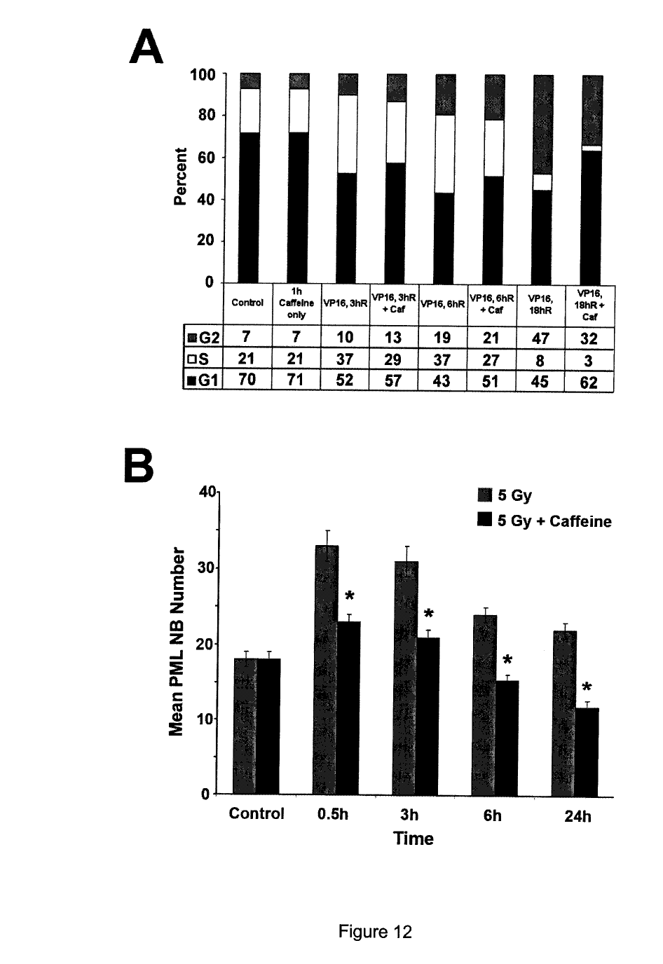

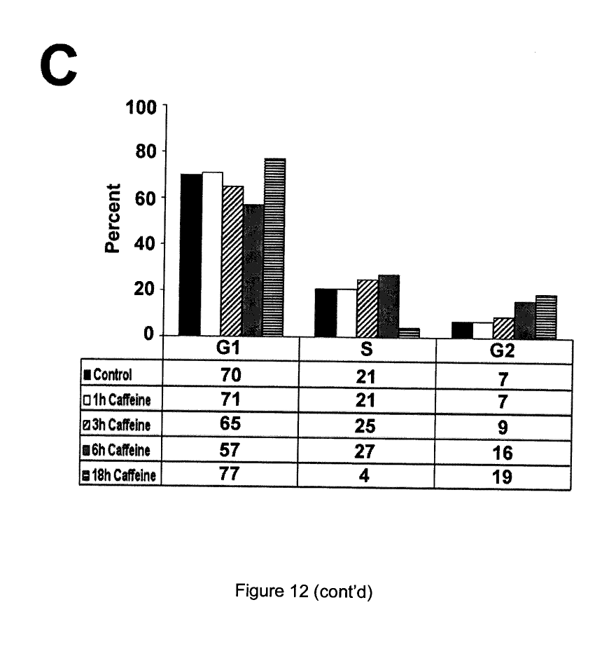

In a further embodiment, rather than determining PML NB numbers in response to the treatment, one may examine any other suitable PML NB property, including PML NB composition changes, morphological changes or behaviour changes as an indicator of PML NB response to the treatment. If the value or profile of values over time of the examined parameter in the treated tumor sample or samples is unchanged or is different relative to the same parameter in control tissue, this indicates that the treatment is not likely to be efficacious. Composition changes of the NB's include, but are not limited to, changes in PML content, changes in PML isoform content, or changes in a PML NB component such as SUMO-1, or post-translational modification of PML or other PML NB components such as phosphorylation, methylation, acetylation, ubiquitination, SUMOylation, biotinylation, and ADP-ribosylation, which may be assayed by methods well known to those of skill in the art. Morphological changes of NB's mean changes from the shape characteristic of NB's in the relevant normal tissue, for example deviation from the spherical shape found in most tissues, such as skin which can be assayed by light or electron microscopy as described herein (FIG. 3). Behavioural changes may be determined by observing live cell dynamics and observing changes of NB mobility, for example deviation from positional stability to unconstrained diffusional movement within the nucleus or fragmentation of bodies into smaller structures as described herein (FIG. 2).

PML NB properties may also be monitored in vertebrate animal models including but not limited to pig, dog, cat, rabbit, rat, guinea pig and mouse, where human tumor cells derived from patients are implanted or injected into the animal creating a tumor, or from endogenous tissues within the animal as result of carcinogen exposure and/or genetic manipulation. These animals are then treated with radiation or chemotherapeutic reagents and the PML NB response may be assayed as described above to ascertain the efficacy of treatment for that tumour.

In a further embodiment, the invention provides a method for monitoring the response of a tumor in a patient to DNA-damaging treatment such as radiation or chemotherapy or monitoring the efficacy of a DNA-damaging agent in tumor treatment.

A biopsy sample of the tumor is taken prior to treatment, the selected treatment is given to the patient and at intervals from at least 30 minutes to several hours, up to and including 48 hours, after treatment, further biopsy samples of the tumor are obtained. All samples are examined for a PML NB property such as PML NB number, as described above. An increase in NB number after treatment relative to that of the pre-treatment biopsy sample indicates that the treatment is having an effect on the tumor. A lack of increase in NB number suggests that the treatment is not effective and should be modified or a new treatment option should be explored.

Alternatively, one can determine another PML NB property such as biochemical composition, including SUMO-1, PML isoforms and post-translational modifications of PML or other PML NB components, as outlined above or morphology, or behaviour, as described above.

As discussed above, if the value or profile of values of the examined NB property is unchanged or is different relative to the same parameter in control tissue, this suggests that the tumor is not normally responsive to the treatment.

The analysis of PML NB number, behaviour and biochemical composition can be made on biopsies as early as 30 min following treatment. This would allow chemotherapy or radiation treatment to be modified within 24 h if the PML NB analysis revealed that the current treatment was ineffectual against the particular tumour. Therefore, the use of PML NB analysis could be used to rapidly tailor treatment for the patient on a tumor by tumor basis.

In a further embodiment, PML NB number of a tumor can be assayed prior to treatment and at intervals such as 30 min, 3 h, 6 h, and 18 h after radiation or DNA-damaging agent treatment as described above and the profile of PML NB increase following irradiation may be used to assay the integrity of DNA repair pathways in the tumor tissue involving NBS1, Chk2, ATR and ATM kinase. The inventors have confirmed that for several key DNA repair pathways, including both the ATM and ATR kinase pathways, PML NB number is a reliable indicator of the ability of the cell to respond appropriately to DNA damage. A failure to increase PML NB number by around 3 hours post-treatment within the cells of the tumor, as compared to the cells within normal tissue of the same type, indicates that these pathways are abrogated.

In a further embodiment, the invention provides a method of monitoring the response of a cell or tissue to stress, such as the stress associated with induction of DNA damage in the cell or tissue, for example due to DNA-damaging agents such as radiation or DNA-damaging chemical compounds or the stress associated with hypoxia or anoxia, ischemia or oxygen/blood deprivation of cellular tissues such as the heart and brain tissue. PML NB number prior to these forms of cellular stress is compared to PML NB number after the stress. Changes in PML NB number after stress may presage clinical outcome or be used to determine the extent of damage within a given organ such as the heart or the brain.

Furthermore, PML NB number, morphology, biochemical composition and behaviour may also be used to assay the efficacy of a combination of cellular stresses such as heat, viral infection, radiation or chemotherapy reagents creating DNA damage, in combination with but not limited to hypoxia or anoxia, and/or the genetic manipulation of DNA repair of tumour suppressor pathways.

The method comprises determining the presence or absence of a change in PML NB number, composition, morphology or behaviour in the cell or tissue, by the techniques described herein.

In the case of cellular stress due to DNA-damaging agents, these agents may have been administered to the cell or tissue deliberately, for example for treatment of cancer, or may have resulted from accidental exposure, such as environmental or work related radiation exposure, for example to radon gas, uranium ore or radioactive waste, or as a result of a nuclear accident, or accidental exposure to DNA-damaging chemical compounds such as aldehydes, and transition metal compounds containing chromium or arsenic.

A change in PML NB number, composition, morphology or behaviour in a post-exposure tissue in the period from about 30 mins to about 72 hours post-exposure, relative to normal values for that tissue, are indicative that the exposure has resulted in DNA-damage.

The degree of elevation of PML NB number, or the degree of change in one or more other PML NB properties, may indicate the severity of the damage to the tissue as a result of the stress. For example, a post-heart attack cardiac muscle biopsy may be examined for change in a PML NB property, as described herein, as an indicator of severity of damage.

The PML protein and by extension PML NBs are implicated in the regulation of both DNA repair processes (Dellaire and Bazett-Jones, 2004) and virtually every major tumour suppressor pathway including p53 (reviewed in Salomoni, and Pandolfi 2002.), Chk2 (Yang et al., 2002), TGF-beta (Lin et al., 2004), and PTEN-AKT (Trotman et al., 2006). Therefore, analyzing PML NBs within tumour biopsies it may enable the detection of oncogenic changes in both DNA repair mechanisms and several of the tumor suppressor pathways using one assay.

In a further embodiment, the invention provides a method for assessing the malignancy of a tumor by determining at least one PML NB property in the tumor and comparing the value of the property in the tumor to the value of that property in normal tissue corresponding to the tumor. The property may be determined in untreated tumor and normal tissue samples, or in tumor and normal tissue samples after exposure to a DNA-damaging agent such as radiation or chemotherapy. The greater the degree of difference in the property between the tumor and the normal tissue, the greater the likely malignancy of the tumor. The PML NB property determined may be at least one of PML NB number, composition, morphology or behaviour, as previously described herein.

Benign and malignant tumors may therefore be distinguished in a particular organ or tissue, for example in skin, brain, muscle, breast, colon and lung.

In addition, each tissue type will have PML NBs exhibiting characteristic properties with respect to number, biochemical composition and the PML protein isoform expression levels. These properties can be used to distinguish cells from tumors that have metastasized from normal tissues that do not carry the same PML NB profile or set of properties, including response to radiation or chemotherapy agents that damage DNA.

The correlation between changes in PML NB properties and degree of malignancy may be confirmed by examination of PML NB properties as described herein in stored tumor samples for which clinical outcome is already known, allowing grading of tumors for clinical outcome by determination of PML NB properties.

Use of PML NB properties as described herein as markers of malignancey may be combined with previously described malignancy markers, for example nuclear antigens such as Ki-67 Gerde et al., 1983), PAN bodies (International Application WO 03/020883) and splicing and ribosomal proteins.

PML NBs make extensive contacts to chromatin in their vicinity, which accounts both for their positional stability during interphase (Eskiw et al., 2004) and their highly dynamic behaviour in S-phase when chromatin topology is altered during DNA replication (Dellaire et al, 2006). Due to their intimate relationship with chromatin, we previously hypothesized that PML NBs might also represent sensors of DNA damage (Dellaire and Bazett-Jones, 2004). In this study we have systematically tested this hypothesis, demonstrating that PML NBs behave as dose-dependent sensors of DSBs.

PML NBs Initially Respond to DSBs by Increasing in Number by Fission.

Chromatin is physically constrained by subnuclear compartments such as the nucleolus and the nuclear lamina (Chubb et al., 2002). Chromatin contacts on the surfaces of the protein-based cores of PML NBs may also serve as chromatin anchoring sites (FIG. 8A). Positional stability of PML NBs and the low rates of chromatin mobility indicate a balance of forces between chromatin and such anchoring sites. Recently, we have shown that chromatin appears to “relax” or decondense in the vicinity of DSBs (Kruhlak et al., 2006). This balance of forces, or tensegrity, may be altered by the introduction of DSBs in chromatin, and could lead to the shearing of the PML NB core as the associated chromatin domains pull away from the body. One outcome of this biophysical response is the supramolecular fission of PML NBs into microbodies immediately following the introduction of DSBs (FIG. 8B). We propose that the biophysical response to changes in chromatin tensegrity is the primary basis for the fission of PML NBs when cells are irradiated at 4° C. (FIG. 2 C), a condition under which biochemical activities like DNA repair and biochemical changes to chromatin and PML NBs are almost entirely blocked (FIG. 8 B). It is unlikely that de novo formation of PML NBs (Everett and Murray, 2005) by the redistribution of PML protein accounts for the initial increase in number of PML NBs, since protein diffusion is very limited below 15° C. (FIG. 2 D). Furthermore, ultrastructural examination of PML NBs in situ following DSB induction with VP16 revealed that the protein core of the PML NB is indeed destabilised in conjunction with structural changes in chromatin (FIG. 4 B). A similar instability of PML NB structure is observed in early S-phase, where the NBs also break apart by fission (Dellaire et al., 2006).

PML NBs are Sensitive Detectors of DNA Damage.

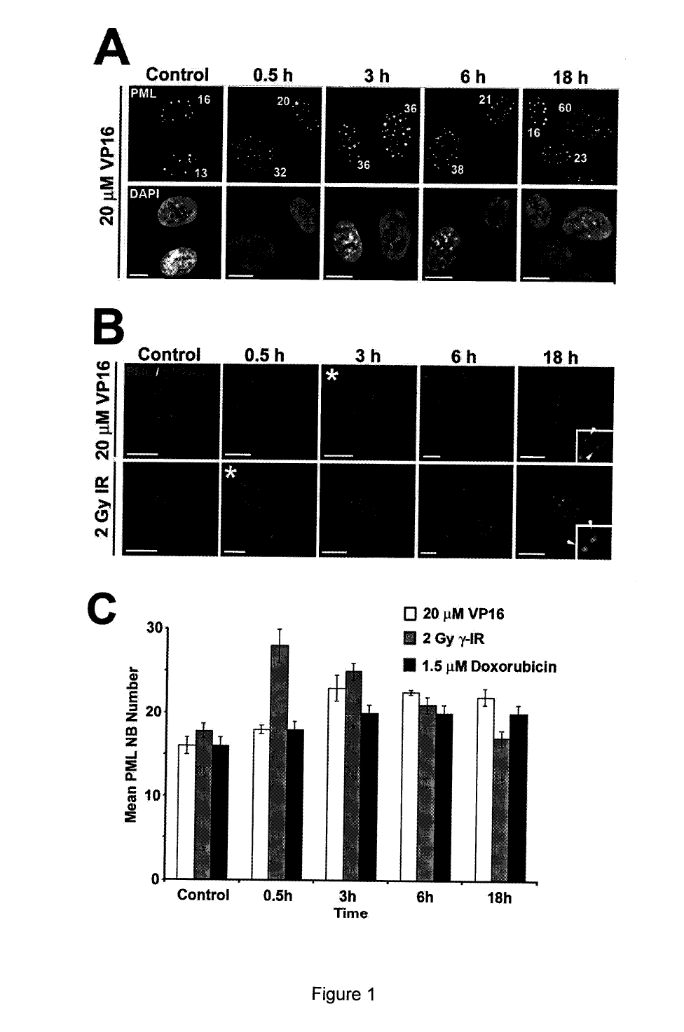

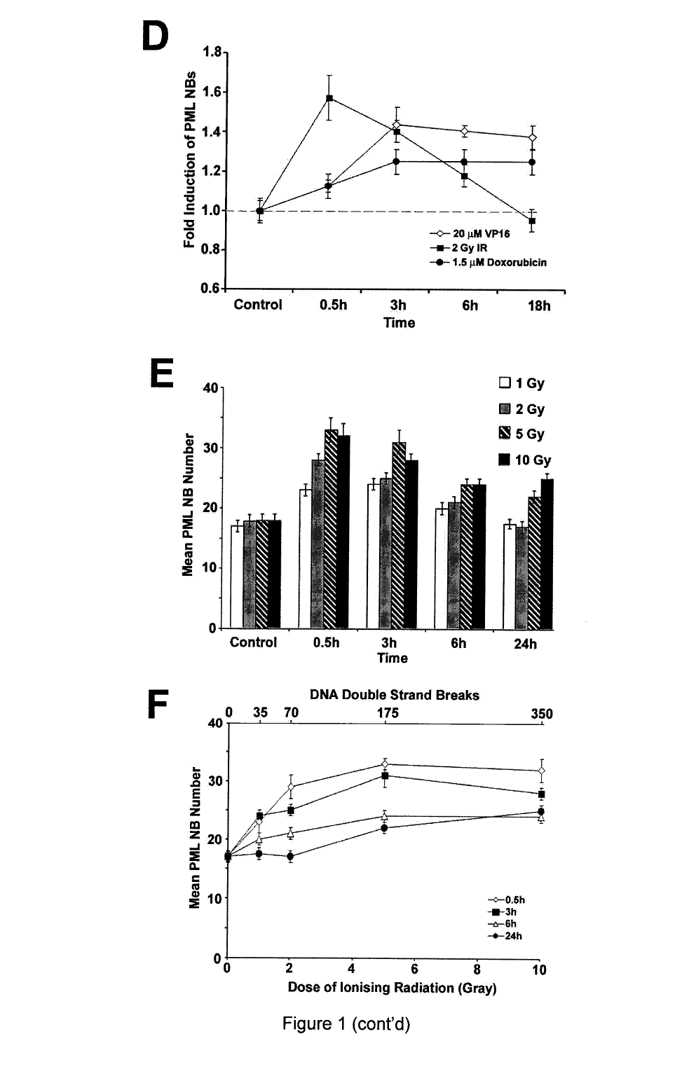

PML NBs behave as DNA damage sensors by increasing in number in response to genotoxic stress. The initial formation of microbodies by a fission mechanism is sensitive to as few as 35 DSBs (i.e. 1 Gy IR). However, PML NB number does not increase in a linear fashion with dose of IR, in contrast to the induction of DSBs and consequently the phosphorylation of H2AX (Rogakou et al., 1999). At high doses of IR (i.e. >5 Gy), PML NB number per cell becomes saturated (FIG. 1F). We can model the relationship of PML NB number to DSBs as a modified power function where body number varies with the power of the dose (FIG. S2A). This relationship holds for PML NB number up to 6 h post-irradiation. In addition, at low doses of IR PML NB number returns to control levels within 24 h, whereas at high doses of IR (i.e. 5 and 10 Gy), PML NB number remains elevated. Doses of IR at 5 and 10 Gy are considered supra-lethal as they inhibit cell growth in clonogenic assays by 90-100% (Bristow and Hill, 2005). Therefore, the failure of PML NB number to return to that of control cells after irradiation may be an indication of either eventual senescence or programmed cell death. PML NB number also remains elevated for extended periods following DNA damage in cells that have impaired cell cycle checkpoints (e.g. due to loss of p53, FIG. 5). Thus, at non-lethal physiological levels of DNA damage PML NB number may provide a simple indicator of the complex response of mammalian cells to DNA damage and the fidelity of the p53-dependent G1/S checkpoint.

Live Cell Analysis Demonstrates that PML NBs are not Recruited to Sites of DNA Damage.

PML NBs appear to associate with single-stranded regions of DNA as well as with foci containing γ-H2AX and/or repair proteins following DNA damage (reviewed in Dellaire and Bazett-Jones, 2004). We also observed the juxtaposition of γ-H2AX foci (FIG. 1B) and the co-localisation of Nbs1 with PML NBs at 6 and 18 h after the induction of DSBs (FIG. S2 A). We also observed at 1.5 h following DSB-induction, the co-localisation and juxtaposition of RPA foci with PML NBs in NHDFs in S— and G2 phase of the cell cycle (FIG. S2 B-C). These data raise the possibility that PML NBs and DNA repair proteins may be able to co-accumulate at sites of DNA damage by the movement of intact PML NBs or by their de novo formation. We tested these assumptions by UV laser-induction of DSBs in defined regions of the nucleus of U-20S cells expressing GFP-PML IV (FIG. 3 A-B). PML NBs in close proximity to the laser-induced DSBs began to lose positional stability and body number dropped as these “liberated” bodies aggregated with each other. However, we did not see the de novo formation of PML NBs along the length of the laser track, as would be expected if PML were co-accumulating at sites of DNA damage. Thus, although PML NBs lose positional stability when chromatin is damaged in their vicinity, they do not form de novo or move to sites of DNA damage. Rather, they are able to move large distances through the nucleoplasm, possibly through spaces or channels created in chromatin by the extensive number of DSBs created along the laser track (Bradshaw et al, 2005). Such long-range movement of PML NBs is consistent with “corral” models of nuclear body movement within the ICD space (Eskiw et al., 2003; Gorisch et al., 2004). Eventually, PML NBs far from the laser track respond by changes in their movement and by microbody formation, suggesting that either global changes in chromatin structure are occurring at later time points following DNA damage and/or ongoing DNA repair processes are required for this phenomenon. The co-localisation of PML NBs with RPA in G2 and S-phase cells at 1.5 h following DSB-induction may indicate a role for PML in homologous recombination events such as sister-chromatin exchange in cooperation with other factors such as BLM (Hu et al., 2001; Rao et al., 2005). The significance of the co-localisation and juxataposition of NBs with γ-H2AX and Nbs1 at late time points following DNA damage (i.e. 6 and 18 h, FIG. S1 A) remains unclear, since >90% of DNA repair has already been completed. Perhaps at these late time points, PML NBs may function as sites of post-translational modification of DNA repair factors at the end of the repair cycle, rather than playing a direct role in the DNA repair mechanisms themselves.

Signaling Pathways Regulating DNA Repair and PML NB Response to DSBs Share Common Factors.

Following the initial rapid formation of PML microbodies by a fission mechanism, further increases in PML NB number accompany ongoing DNA repair. This can be explained by the sensitivity of PML NBs to the topological state of chromatin (Dellaire et al., 2006), which is affected by chromatin remodeling associated with DNA repair (Lydall and Whitehall, 2005). Therefore, mutations in components of the DNA repair response could also abrogate the breakdown of PML NBs into microbodies. ATM and ATR kinases are key regulators of the cellular response to DNA damage, which may play partially redundant roles since they share many of the same substrates such as Chk2, Brca1 and p53 (reviewed in Shiloh, 2001 and Pommier et al., 2005).

We addressed the role of the ATM and ATR DNA repair pathways in regulating the increase in PML NB number in response to DSBs, by using both repair deficient cell lines and chemical inhibition of ATM, ATR and Chk2 kinase. AT cells expressing mutant ATM or NHDF treated with wortmannin, an inhibitor of ATM, demonstrated a similar delay in the increase in PML NBs in response to DSBs, which was most significant at 3 h following VP16 treatment (FIG. 6 A-B). A similar delay in the increase in PML NB number occurred at 3 h in NHDFs treated with a Chk2-specific kinase inhibitor (FIG. 6A) and in Chk2−/−MEFs (FIG. 7 A). We also found that loss of Nbs1, which is a member of the MRN DNA damage sensing complex (D'Amours and Jackson, 2002) and is required for activation of Chk2 by ATM in response to low levels of DNA damage (Buscemi et al., 2001), caused a significant delay in the increase in PML NB number in response to VP16 (FIG. 6B and FIG. 7B). Therefore, the inhibition of PML microbody formation in NBS or AT cells at 3 h post-etoposide treatment (FIG. 6B) may result in part from a failure to activate Chk2 in response to DSBs. In addition, chromatin topology changes following DNA damage may be subject to regulation by ATM kinase (Y. Shiloh, personal communication). Therefore, ATM's role in regulating PML NB behaviour following DNA damage could be two fold: a) through the activation of Chk2, and b) via modulation of chromatin topology following DNA damage.

NBS cells also had many fewer PML NBs initially at 30 min following VP16 treatment as compared to ATM- or Chk2-deficient cells. In contrast, Mre11-deficient ATLD cells showed an initial increase in PML NB number similar to NHDFs followed by a significant reduction in PML NB number at all other time points. Since Nbs1 and Mre11 are in the same DNA damage-sensing complex, the basis for this discrepancy between NBS and ATLD cells with regard to PML NB induction is unclear. An intriguing possibility is that Nbs1 may play an earlier and separate role in regulating the increase in PML NB number in response to DSBs beyond the activation of Chk2.

PML NB number following VP16 treatment was inhibited to an even greater extent in Seckel syndrome cells deficient in ATR (O'Driscoll et al., 2003) than for cells deficient in ATM, Chk2 or Nbs1 (FIG. 6B). In addition, the dominant-negative inhibition of ATR kinase by expression of a kinase-dead mutant of ATR (ATR-DN, FIG. 7 C) in U-2 OS cells also inhibited the increase in PML NB number in response to DSBs. The most dramatic inhibition, however, occurred when we inhibited both ATM and ATR with caffeine (FIG. 6A). At all time points following DSB induction PML NB number remained at or below that of untreated cells. The additive effect of inhibition of ATM- and ATR-dependent pathways on the increase in PML NB number in response to DSBs by caffeine suggests they act in parallel pathways, consistent with their redundant roles in regulating DNA damage checkpoints (Shiloh et al., 2001). Although the initial biophysical response of PML NBs is dependent on changes in the tensegrity and topological state of chromatin associated with DSB-induction and DNA repair, it remains an intriguing possibility that ATR or Chk2-dependent phosphorylation of PML and PML NB constituents (Bernardi et al., 2004; Yange et al., 2002) may also contribute to NB instability (summarized in FIG. 8C). Other forms of DNA damage, such as UV-irradiation (Seker et al., 2003), also appear to destabilise PML NBs. Since ATR kinase is strongly activated by UV-irradiation, DNA SSBs, and drugs that induce replication fork stalling (Shiloh et al., 2001), it is likely that the increase in PML NB number seen in response to these cellular stresses is also regulated by ATR kinase. We are currently testing this hypothesis.

In summary, we have demonstrated that PML NBs are indeed dynamic sensors of DNA damage that respond to DNA DSBs by increasing in number primarily by a fission mechanism. The DSB-dependent increase in PML NBs occurs first as a biophysical response to changes in chromatin. However, soon after DSB induction PML NB number becomes sensitive to cell cycle checkpoint control and on-going DNA repair, being regulated by ATR kinase and to a lesser extent ATM kinase, possibly through Nbs1-dependent activation of Chk2 kinase. This dynamic behaviour of PML NBs in mammalian cells provides an exceptional pathological marker for cellular health, cell cycle progression and the integrity and stability of the genome.

### EXAMPLES

The examples are described for the purposes of illustration and are not intended to limit the scope of the invention.

Methods of chemistry, histology, molecular biology protein and peptide biochemistry and immunology referred to but not explicitly described in this disclosure and examples are reported in the scientific literature and are well known to those skilled in the art.

### Methods and Materials

Cell Culture and drug treatments. Cell lines used in this study: NDHFs (GM05757, Coriell Cell Repository); human AT fibroblasts (a.k.a. AT5B1, GM05823; Coriell); human NBS-T fibroblasts (gift of J. Lukas, Danish Cancer Society, Copenhagen, Denmark); human ATLD fibroblasts (gift of Y. Shiloh, Tel Aviv University, Tel Aviv, Israel) Saos-2 (American Tissue Culture Type Collection (ATCC), HCT116 and p53 null HCT116 isogenic cells (gift of Dr. B. Vogelstein, Department of Oncology, John Hopkins University, Baltimore, Md.); ATR-DN and ATR-WT cells (gift of Dr. Paul Nghiem, University of Washington Medical School, Seattle, Wash.); Seckel syndrome cells (GM18366, Coriell); Chk2−/−MEFs and isogenic WT MEFs (gift of Dr. T. Mak, University of Toronto, Toronto); and isogenic and U2OS cells stably expressing GFP-PML IV (gift of J. Taylor, U. Wisconsin, Milwaukee, Wis.). NBST-pBabe and NBST-pBabe-NBS1 cells lines were generated by retroviral transduction of NBS-T fibroblasts using either pBabe-Puro alone or encoding full length human NBS1 (gift of J. Lukas), respectively. To generate DSBs, cells were treated with 20 μM etoposide (VP16; Sigma) or 1.5 μM doxorubicin (Sigma) for 30 min, washed 2× in phosphate buffered saline (PBS)(Wisent), and left to recover for the indicated time. We determined that 20 μM VP16 for 30 min was equivalent to ˜2 Gy of IR by both clonogenic (data not shown) and the neutral comet assay (FIG. S2 B). Alternately, asynchronous cell cultures were exposed to whole-cell ionizing radiation (IR)(dose range, 0-20 Gy) using a ¹³⁷Cs irradiator (MDS Nordion, Ottawa, Canada) at 1 Gy/min (on ice, aerobic conditions). For kinase inhibition studies, cells were incubated with growth medium supplemented with 20 μM Wortmannin (Sigma), 5 mM caffeine (Sigma), 50 μM LY294002, or 10 μM Chk2 inhibitor II (EMD Bioscience; Arienti et al., 2005) for 30 min, prior to addition of VP16 or exposure to IR. Cells were then maintained in growth medium containing kinase inhibitors for the indicated time. For the inhibition of protein synthesis, cells were treated with 150 μg/ml of cycloheximide (CHX)(Sigma) for 30 min prior to treatments and maintained in CHX until processed for light microscopy.

IF detection of proteins, live-cell imaging and fluorescence recovery after photobleaching (FRAP). Cells grown on cover slips were treated with or without kinase or protein synthesis inhibitors, prior to DSB induction (etoposide or IR), fixed and processed for IF as previously described (Dellaire et al., 2006). Primary antibodies used in this study: rabbit anti-PML (Chemicon, AB1370); rabbit anti-Sp100 (Chemicon, Ab1380); mouse anti-RPA (Calbiochem, RPA34-20); mouse anti-phospho-Histone H3 (ser10) (Upstate, MC463); rabbit anti-Cyclin A (Santa Cruz, sc-751); mouse anti-SUMO1 (Zymed, GMP-1); mouse anti-γ-H2AX (Upstate, JBW301). Secondary antibodies conjugated to Cy3 and Cy5 were obtained from Jackson Labs and secondary antibodies conjugated to Alexa488 were obtained from Invitrogen Molecular Probes. DNA was stained with 4,6 diamidino-2-phenylindole (DAPI)(Sigma) in mounting media containing 90% glycerol and 1 mg/mL paraphenylenediame (PPDA)(Sigma). Fluorescence micrographs of fixed cells were collected using an HCX PL APO CS 63×/1.32 N.A. oil immersion objective lens (Leica Microsystems) on a Leica DMR2 upright fluorescence microscope fitted with a Hamamatsu Orca camera. OpenLab 3.5.1 software (Improvision) was used for image acquisition. Live-cell imaging and FRAP analysis of GFP-PML IV in U-2 OS cells was carried out, as previously described (Dellaire et al., 2006). ImageJ v1.33 (NIH) and Photoshop 7.0 software were used for image processing and analysis.

Statistical analysis. To determine mean PML NB number, maximum intensity projections of multiple focal planes were generated for the IF localization of PML using OpenLab 3.5.1 software (Improvision). PML NBs were counted in a minimum of 30 cells per time point and the body number/cell was normalised for nucleus size. Normalisation of body number was accomplished by multiplying the ratio of the area of each nucleus divided by the average area of a nucleus in a given data set. This calculation was necessary to account for the variability in PML NB number due to cell cycle phase or ploidy (Dellaire et al., 2006). However, in normal diploid cell lines this calculation will not affect the mean body number/cell but will reduce statistical variability between data sets. Each experiment was repeated in triplicate and the mean PML NB number was used directly or divided by the mean number of bodies in the control (untreated) to give the fold induction of PML NBs. Error analysis for triplicate experiments is expressed as the standard error of the mean (s.e.m.); where s.e.m.=s.d./√3. For all other experiments error analysis was expressed as simply standard error (s.e.). Data sets of PML NB number/cell exhibit a normal distribution, therefore statistical significance between data sets was derived using the Student's T-test for pair-wise analysis using MS Excel and by analysis of variance (ANOVA) for testing the significance of IR dose on PML NB number using on-line statistical tools available from http://www.physics.csbsju.edu/stats/anova.html. Curve fitting for PML NB induction versus dose of IR was accomplished on-line using tools available from http://zunzun.com.

Induction of subnuclear DNA damage by UV-laser treatment. Cells grow on coverslips were incubated for 5 min in growth medium containing 0.5 μg/ml of Hoechst 333258 to sensitize cells to the UV-laser-induced damage. An LSM 510 confocal microscope (Carl Zeis MicroImaging), equipped with an argon laser (488 nm) and tunable multiphoton laser (Chameleon, Coherent Inc.) capable of effective wavelengths in the UV range, was used to image cells and to generate UV-laser induced damage using a Plan-Apochromat 63×/1.40 N.A. oil immersion objective lens (Carl Zeis MicroImaging). Laser damage was accomplished by selecting an ROI within a cell and bleaching the ROI using the tunable laser set at 790 nm (effective λ=390 nm) and 20% power for a 200 ms pulse. At 20% power, the laser generates 7-8 mW, which translates to a cellular dose of ˜80 Gy (Bradshaw et al., 2005). Images were then collected immediately after the bleach using the argon laser at 50% power and 10% transmittance. Cells were maintained at 37° c. during live cell imaging using a heated stage (Bioptechs)

Correlative Microscopy and Electron Spectroscopic Imaging—Samples were prepared and sectioned for correlative microscopy and electron spectroscopic imaging (ESI) as previously described (Eskiw et al., 2003; Dellaire et al., 2004). Nitrogen and phosphorus maps were collected using an FEI Technai 20 transmission electron microscope fitted with an electron imaging spectrometer (Gatan). Immuno-gold labeling was accomplished using a secondary antibody conjugated to Ultra-small nanogold (donkey anti-rabbit, EMS, Cat. 25100).

Cell Culture requirements. NDHFs (GM05757, Coriell Cell Repository), human Seckel syndrome fibroblasts (GM18366, Coriell) and human AT fibroblasts (GM05823; Coriell) were cultured in α-MEM (Wisent)+15% fetal bovine serum (FBS)(Sigma). Human AT fibroblasts (AT5B1, GM05823; Coriell), human NBS-T fibroblasts (gift of J. Lukas, Danish Cancer Society, Copenhagen, Denmark), human ATLD fibroblasts (gift of Y. Shiloh, Tel Aviv University, Tel Aviv, Israel), Saos-2 (American Tissue Culture Type Collection (ATCC), HCT116 and p53 null HCT116 isogenic cells (gift of Dr. B. Vogelstein, Department of Oncology, John Hopkins University, Baltimore, Mass.) were cultured in DMEM 10% FBS. ATR-DN and ATR-WT cells (gift of Dr. Paul Nghiem, University of Washington Medical School, Seattle, Wash.); Chk2−/−MEFs and isogenic WT MEFs (gift of Dr. T. Mak, University of Toronto, Toronto), were cultured in tetracycline-free DMEM 10% FBS. Induction of flag-tagged ATR-DN in these cells was accomplished by addition of doxycycline (Sigma) to a final concentration of 1.5 μg/ml in the growth medium for 24 or 36 h. U-2 OS cells stably expressing GFP-PML IV (gift from J. Taylor, U. Wisconsin, Milwaukee, Wis.) were cultured in DMEM 10% FBS+1 mg/ml 1.6 mg/ml G418 (Wisent), respectively.

### Immunofluorescence Microscopy and BrdU Labelling

Cells were prepared for immunolabelling as described (Eskiw et al., 2003). To label with BrdU, cells were pulsed with 20 μM BrdU for 30 minutes, fixed in methanol for 30 minutes at −20° C. The methanol was removed and 4N HCl was added to the cells for 2 minutes at room temperature (RT). Cells were washed 3× with PBS at RT. Primary antibodies used were: mouse anti-PML, clone 5E10 (1:10)(a gift from R. van Driel, University of Amsterdam, The Netherlands), rabbit anti-PML (1:1000)(Chemicon, AB1370); rabbit anti-Sp100 (1:500)(Chemicon, Ab1380); mouse anti-RPA (1:200)(Calbiochem, RPA34-20); mouse anti-phospho-Histone H3 (ser10) (1:500)(Upstate, MC463); rabbit anti-Cyclin A (1:200)(Santa Cruz, sc-751); mouse anti-SUMO1 (1:20)(Zymed, GMP-1); mouse anti-y-H2AX (1:500)(Upstate, JBW301); mouse anti-BrdU (1:10)(BD Biosciences, clone B44). Secondary antibodies used were donkey anti-mouse or rabbit Cy3 and Cy5 (Jackson Laboratories). For electron microscopy, donkey anti-rabbit antibody conjugated with Ultra-small nanogold (Electron Microscopy Sciences (EMS), Cat. 25100) was used. For fluorescence microscopy, coverslips were mounted on glass slides using 90% glycerol/PBS containing 1 mg/ml paraphenlenediamine and 1 μg/ml 4′,6-Diamidino-2-phenylindole (DAPI). Images were collected on a Leica Microsystems DMRA2 upright microscope equipped with a Hamamatsu ORCA-ER camera using the 63×1.32 NA oil-immersion objective. For spinning disc laser confocal microscopy of living cells, images were acquired using a Leica DMIRE2 inverted microscope equipped with a Hamamatsu ORCA-AG camera, a Wave FX spinning disk confocal unit (Quorum Technologies), and an argon laser (Melles Griot). OpenLab 3.5.1 (Improvision) software was used to collect images, and the images were processed with Photoshop 7.0 (Adobe). Line scans were performed using ImageJ (NIH) software.

### Western Analysis

Cells were grown in six-well dishes and 2×SDS-PAGE loading buffer was added to the cells. Protein samples were separated by 10% SDS-PAGE and transferred onto nitrocellulose (Amersham Biosciences). PML, Sp100 and actin proteins were detected using rabbit anti-PML (AB1370, Chemicon), rabbit anti-Sp100 (AB1380, Chemicon) and mouse anti-actin (Sigma) antibodies. Secondary labeling was conducted using goat anti-rabbit HRP (Cell Signaling) and goat anti-mouse HRP (Sigma). Bands were detected using an ECL kit and quantified using Fluor Chem 2.0 (Alpha Innotech Corporation) software. All isoforms of PML above 72 kDa were used for quantification of PML protein levels.

### Flow Cytometry

Cells were harvested and pelleted. The pellet was resuspended in 50 μl HBSS with 2% FBS. Cells were fixed with 1 ml ice-cold 80% ethanol for 30 minutes on ice. Cells were pelleted and resuspended in HBSS containing 0.1 mg/ml propidium iodide (Sigma) and 0.6% NP-40 to final concentration of 2×10⁶ cells/ml. An equal volume of 2 mg/ml RNase in HBSS was added and incubated at room temperature in the dark for 30 minutes. This solution was then filtered through a 40 μm nylon mesh (BD Biosciences) and keep on ice until analysis. Cells were analyzed on a FACscan flowcytometer (BD Biosciences). Data analysis was performed using FlowJo 6.0 (Tree Star).

### Single-Cell Neutral Comet Assay

Single-cell suspensions were mixed with 75 μL of 0.5% low-melting agarose at 37° C. and spread on a 1% agarose precoated slide. Slides were then incubated in Proteinase-K solution for 60 minutes at 37° C. followed by incubation in ice-cold lysis buffer (2.5 mol/L NaCl, 100 mmol/L EDTA, 10 mmol/L Trizma base, 10% DMSO, 1% Triton-X) overnight. After lysis, the slides were placed in horizontal electrophoresis tanks filled with electrophoresis buffer (1×TBE, pH 8.0) for 20 minutes and then subjected to electrophoresis at 25 V/30 to 45 mA for a further 20 minutes. After electrophoresis, the slides were washed 3× in wash buffer (0.4 mol/L Tris-HCl, pH 7.5), air-dried, and stained with ethidium bromide (2 μg ml⁻¹) before scoring. The Olive Tail Moment (Olive et al., 1990), which is the relative amount of fragmented DNA contained within the Comet's tail compared with the nonfragmented DNA within the Comet head, was determined by fluorescent image analysis (Northern Eclipse software) as a measure of residual DNA breaks over time following irradiation or etoposide treatment.

### Example 1

### The Increase in PML NB Number in Response to DSBs is Sensitive, Rapid and Dose-Dependent

The response of PML NBs to DSBs in a normal human diploid fibroblast (NHDF) cell line GM05757 was assessed, using ionising radiation (IR), etoposide (VP16) and doxorubicin (FIG. 1). IR generates both single-strand breaks (SSBs) and DSBs in DNA, whereas, the topoisomerase II-inhibitors VP16 and doxorubicin create primarily DSBs (reviewed by Kurz and Lees-Miller, 2004). PML NBs were counted in maximum intensity Z-projections of individual cells. PML NB number increased following DSB induction (FIG. 1A). Furthermore, we found that the time point associated with the highest number of PML NBs coincided with the peak of H2AX phosphorylation (γ-H2AX; FIG. 1B), an event that occurs on chromatin surrounding DSBs (Rogakou et al., 1999). Maximum PML NB number correlated with peak γ-H2AX signal regardless of the method of DSB-induction, suggesting that the increase in PML NB number is coupled to DSB formation. PML NB induction was most rapid for IR, peaking at 30 min post-IR (FIG. 1C-D). In contrast, γ-H2AX signal and PML NB number peaked later at 3 h following treatment with VP16 or doxorubicin (FIG. 1B-D). Consistent with previous studies of PML NB association with γ-H2AX and components of the Mre11/Rad50/Nbs1 (MRN) complex (Carbone et al., 2002; Xu et al., 2003), we observed foci of γ-H2AX and Nbs1 that partially co-localised with or were juxtaposed to PML NBs between 6 and 18 h following DSB induction (FIG. 1 B and FIG. 9A). In contrast, we observed a much earlier co-localisation and juxtaposition between foci of RPA and PML NBs at 1.5 h after DSB-induction, which persisted for up to 18 h (FIG. 9B). Following etoposide treatment only a sub-population of cells in S- and G2-phase develop RPA foci in NHDFs. Therefore, the association of PML NBs with RPA foci following DNA damage is restricted to S- and G2-phase of the cell cycle (FIG. 9C).

We then tested whether the increase in PML NB number in response to DSBs is dose-dependent by treating cells with varying doses of IR from 0-10 Gy (FIG. 1E-F). We found that at doses as low as 1 Gy (i.e. producing ˜35 DSBs (Bristow and Hill, 2005)), PML NBs increased in number in NHDFs, and the response was dose-dependent, based on analysis of variance between our data sets (Table 1). In contrast to low doses of IR where the number of PML NBs returned to baseline levels by 24 h post-irradiation, at higher doses of 5 and 10 Gy, PML NB numbers remained elevated for an extended period of time (FIG. 1E-F). Therefore, following low doses of IR, the increase in PML NB number following DNA damage appears to be reversible. When PML NB number is plotted versus dose of IR, body number appeared to reach a plateau at doses of 5 Gy or above for all time points with the exception of 24 h (FIG. 1F). Therefore, PML NB number varies with the power of the dose of IR and can be described by the modified power function, y=a*b^{X}+c; where y is the number of PML NBs, and x is the dose of IR in Gray (FIG. S2A). Thus, the increase in PML NB number in response to DSBs is rapid, sensitive to sub-lethal levels of DNA damage and is dose-dependent.

### Example 2

### PML Protein and NB Dynamics in Response to DSBs

The dynamics of PML NBs following DSB induction with VP16 was examined by live cell analysis of U-2 OS cells stably expressing PML isoform IV (FIG. 2). We found that within 5 min after addition of VP16, new and smaller PML-containing structures began to appear adjacent to the larger PML NBs that were present before treatment (FIG. 2A). These new bodies, which we term microbodies, arise from pre-existing PML NBs by a supramolecular fission mechanism, as confirmed by spinning-disc confocal microscopy (FIG. 2B). This fission mechanism is similar to that observed for new PML NB formation in early S-phase (Dellaire et al., 2006), as PML NB biochemical composition was initially indistinguishable between microbodies and the larger parental PML NBs with respect to Sp 100 and SUMO-1 content (FIG. 11A-B). However, although Sp100 levels at PML NBs did not change over the time course observed (FIG. 11A), we did notice a reproducible drop in SUMO-1 levels in PML NBs at 3 h after VP16 treatment (FIG. 11B-C). Over-expression of SUMO-1 dramatically reduced PML NB number (FIG. 11D) resulting in enlarged bodies that showed reduced or delayed increase in PML NB number in response to DSBs (FIG. 11E). It is unclear if over-expression of SUMO-1 is directly or indirectly responsible for the stabilization of PML NBs in our experiments since sumoylation is implicated in many biological pathways, including DNA repair (Gill, 2004).

PML microbodies also formed immediately following irradiation with doses as low as 1 Gy of IR (data not shown) and an increase in PML NB number was seen even when cells were irradiated and fixed on ice to prevent diffusional movement of PML protein or ongoing DNA repair (FIG. 2C). At temperatures below 15° C., PML protein diffusion is very limited as confirmed by fluorescence recovery after photobleaching (FRAP) analysis (FIG. 2D). Interestingly, we also observed a 10% difference in the maximum fluorescence recovery between control and etoposide-treated cells, consistent with a larger immobile fraction of PML protein in bodies following DNA damage (FIG. 2D).

We next examined the behaviour of PML NBs in the vicinity of site-specific DSBs induced by UV-laser irradiation (FIG. 3A-B). Within 5 min of the induction of DSBs, PML NBs in the vicinity of the laser track began to move and coalesce (FIG. 3A, white arrow heads). This process continued for over 20 min resulting in a drop in PML NB number from 21 to 17 NBs but did not affect PML NBs distal to the laser track (FIG. 3A). At later time points, however, even PML NBs far from the laser track lost their positional stability. Imaging of cells in the absence of UV-laser microbeam irradiation did not affect the mobility or number of PML NBs. Continuous imaging by laser scanning confocal microscopy (LSM) following DNA damage did not reveal microbody formation, likely due to photo-bleaching and the loss of visibility of small PML-containing structures. However, after fixation and immunofluorescence detection of PML and γ-H2AX by wide-field microscopy (WFM) 1 h after photo-induction of DNA DSBs, it was apparent that the DNA damage was confined to the laser track and that PML NB number had increased from 17 to 36 PML NBs (FIG. 3B). Although WFM is generally more sensitive than LSM for the detection of PML microbodies, we found that LSM was sufficient to detect greater than 90% of bodies within a focal plane (data not shown). Therefore, the increase in number of PML NBs at 1 h after DSB-induction is primarily due to microbody formation.

### Example 3

### Structural Destabilization of PML NBs Correlates Closely with Topological Changes in Chromatin Following DNA Damage

To address the ultrastructural changes in PML NBs following DNA damage, we employed immuno-gold detection of PML with correlative light and electron spectroscopic imaging (LM/ESI, Dellaire et al., 2004)(FIG. 4). Using LM/ESI we observed that in control NHDFs, PML NBs exhibit radial symmetry and make extensive contacts with the surrounding chromatin (FIG. 4A). Upon treatment with VP16, we found that PML NBs lose their radial symmetry and make fewer contacts with the surrounding chromatin fibres. We also observed “microbody-like” structures, identified by immunogold detection of PML, adjacent to chromatin in the vicinity of larger “parental” PML NBs (FIG. 4B-C). A much larger interchromatin domain (ICD) space was also apparent in cells treated with VP16 (i.e. black spaces outside of chromatin in FIG. 4). These changes in both chromatin and PML NBs are reminiscent of those seen in cells entering S-phase (Dellaire et al., 2006). Based on these results, we suggest that the introduction of DSBs results in topological changes in chromatin linked to PML NBs, which destabilizes the PML NB core.

### Example 4

### The Increase in PML NB Number in Response to DSBs Does not Require p53 or On-Going Protein Translation

PML protein levels can increase following treatment with ionising radiation in a p53-dependent manner (de Stanchina et al., 2004). Therefore, we examined the PML NB response to DNA DSBs in NHDFs with inhibition of PML protein synthesis by treatment with cycloheximide (CHX) and in cells that lack a functional p53 pathway (i.e. null-p53 human Saos-2 osteosarcoma cells and paired HCT116 cell lines isogenic save for p53 protein) (FIG. 5). We found that PML protein levels increased slightly, by 1.3-fold at 4 h after VP16 treatment, reaching 1.8-fold by 12 h (FIG. 5A). As expected, CHX treatment inhibited the DNA damage-dependent increase in PML protein levels at 12 h but had little effect at 4 h, suggesting that post-translational regulation of PML protein levels may occur at this earlier time point. We found that inhibition of protein synthesis or loss of p53 function did not prevent the initial increase in PML NB number (i.e. at 30 min and 3 h) in response to DSBs (FIG. 5B-C). Loss of p53 in the HCT116 cell background actually appeared to enhance the increase in PML NB numbers at 30 min following VP16 treatment (FIG. 5C), perhaps due to further genome instability from a concurrent loss of the mismatch repair factor MLH1 (Koi et al., 1994).

CHX-treated NHDFs exhibited a higher number of PML NBs initially, compared to untreated cells, and body number returned to control levels much earlier than in untreated NHDFs (FIG. 5B). In contrast, PML NB number in VP16-treated Saos-2 cells continued to increase over time (FIG. 5B). PML NB number is affected by cell cycle progression and increases in early S-phase (Dellaire et al., 2006). Fluorescence activated cell sorting (FACS) analysis revealed that after VP16 treatment, NHDFs showed a marked accumulation in G1 and G2 phase of the cell cycle by 18 h (FIG. 12A). Therefore, differences in the number of cells in S-phase at the late time points (i.e. 6 and 18 h) might account for the continued increase in PML NBs observed in the G1/S checkpoint-deficient Saos-2 cells. We examined this possibility by detecting BrdU incorporation at 18 h post-VP16 treatment to determine the fraction of cells replicating DNA (FIG. 5D). We estimate that ˜40% of the Saos-2 cells were in S-phase, compared to only 5-6% of NHDFs, and no BrdU incorporation was observed in NHDFs treated with CHX. Therefore, at early time points the increase in PML NB number is independent of both new protein synthesis and p53. However, at later time points following DNA damage, PML NB number is sensitive to loss of p53 due to abrogation of the G1/S checkpoint, and as a result, PML NB number continues to increase as cells enter S-phase inappropriately.

### Example 5

### The Increase in PML NB Number in Response to DSBs is Abrogated by Caffeine and Inhibited by the Loss of Function of NBS1, ATM, Chk2 and ATR Kinase

Since ATM, ATR and Chk2 kinase are key regulators of the cellular response to DNA damage (Shiloh, 2001; Pommier et al., 2005), we examined whether chemical inhibition of these kinases might affect the response of PML NB to DSBs in NHDFs (FIG. 6A). Inhibition of ATR and ATM kinase by 5 mM caffeine had an inhibitory effect on the increase of PML NB number following VP16 treatment at all time points (p<0.0001, FIG. 6A). Similarly, caffeine significantly reduced the response of PML NBs to 5 Gy of IR (p<0.001, FIG. 12B). This effect was not due to caffeine-dependent changes in the cell cycle profile, since PML NB number did not change when cells were pre-treated with caffeine for 30 min before induction of DSBs (FIG. 6A), and only prolonged treatment with caffeine had an effect on the cell cycle profile of NHDFs with or without VP16 treatment (see 18 h; FIGS. 12A and C). Pre-treatment of NHDFs with the Chk2 kinase inhibitor II (Arienti et al., 2005) did not affect the initial increase in PML NBs in response to VP16 but did significantly reduce the number at 3 h, compared to cells treated with VP16 in the absence of inhibitor (p<0.0003, FIG. 6A). Similarly 20 μM wortmannin, which strongly inhibits DNA-PK and ATM kinase but weakly inhibits ATR, had a significant effect on PML NB number only at 3 h following VP16 treatment (p<0.001, FIG. 6A). In contrast, the DNA-PK inhibitor LY2942002 had little effect on PML NB number in response to DSBs.

We also treated a number of repair-deficient cell lines with VP16 to compare the PML NB response after DNA damage. As with chemical inhibition of ATM, AT cells, which are deficient in ATM, showed a significant inhibition of PML NB number increase only at 3 h following VP16 treatment (p<0.02, FIG. 6B), after which PML NB number actually increased beyond that expected for NHDFs at 6 h. ATLD cells expressing mutant Mre11 (Stewart et al., 1999), a component of the DNA damage sensor known as the MRN complex (Mre11/Rad50/Nbs1)(D'Amours and Jackson, 2002), showed an initial increase in PML NB number following VP16 treatment at 30 min similar to NHDFs. At time points 3 h and later, however, the increase in PML NB number was inhibited by loss of Mre11 function (p<0.0001, FIG. 6B). The increase in PML NB number following induction of DSBs was significantly inhibited at all time points observed in NBS cells, deficient in NBS1, also a member of the MRN complex (Carney et al., 1998), and was profoundly inhibited in Seckel syndrome cells, deficient in ATR kinase (p<0.0001, FIG. 6B) (O'Driscoll et al., 2003).

Since the concentration of Chk2 inhibitor used in our experiments could have residual effects on other kinases (i.e. <25% inhibition of a panel of 35 kinases, Arienti et al., 2005), we wished to confirm the role of Chk2 in regulating the response of PML NBs to DSBs using a genetic mouse model. As predicted from our inhibitor data, Chk2−/−MEFs had an abrogated PML NB response to DSBs, compared to isogenic wild type MEFs at 3 h following VP16 treatment (p<0.001, FIG. 7A). Similarly, reconstitution of NBS cells with wild type human NBS1 by retroviral transduction resulted in a robust increase in body number at 3 h following VP16 treatment, confirming a role for NBS1 in regulating the PML NB response to DSBs (FIG. 7B). Finally, we further characterised the role of ATR kinase in regulating the response of PML NBs to DSBs by VP16 treatment of U-2 OS cells expressing an inducible dominant negative mutant of ATR (kinase dead ATR-DN, FIG. 7C)(Nghiem et al., 2001). Induction of ATR-DN in these U-2 OS cells for 24 h prior to VP16 treatment significantly inhibited PML NB induction at 30 min, 3 h (p<0.0001) and 6 h post-treatment (p<0.001, FIG. 7C). Interestingly, PML NB numbers continued to rise in U-2 OS cells expressing the ATR-DN protein, possibly due to extensive genome instability and eventual apoptosis associated with prolonged expression of this protein. Even within the population of U-2 OS cells expressing the ATR-DN protein, high expression correlated with reduced PML NB number, compared to low expressing cells at 3 h following VP16 treatment (FIG. 7C).

TABLE 1
Analysis of variance (ANOVA) test of the dependence
of PML NB number on the dose of ionising radiation
(IR) in human fibroblasts

| Time | Source of | Sum of | Degrees of | | | |
|---|---|---|---|---|---|---|
| Post-IR | Variation¹ | Squares | Freedom | MS² | F-Ratio³ | p-value⁴ |
| 0.5 h | BG | 5640 | 4 | 1410 | 25.05 | <0.0001 |
| | WG | 8161 | 145 | 56.28 | | |
| 3 h | BG | 3102 | 4 | 775.6 | 17.51 | <0.0001 |
| | WG | 6423 | 145 | 44.30 | | |
| 6 h | BG | 875.9 | 4 | 219 | 5.443 | 0.0004 |
| | WG | 5834 | 145 | 40.23 | | |
| 24 h | BG | 1488 | 4 | 372.1 | 11.69 | <0.0001 |
| | WG | 4615 | 145 | 31.83 | | |

¹Source of variation = between groups (BG) or within groups (WG)
²MS = mean squares
³The F-Ratio is calculated by MS_{BG}/MS_{WG}
⁴The null hypothesis that PML NB number does not vary with dose of IR is rejected if the p-value is <0.05

### REFERENCES

Arienti, K. L., A. Brunmark, F. U. Axe, K. McClure, A. Lee, J. Blevitt, D. K. Neff, L. Huang, S. Crawford, C. R. Pandit, L. Karlsson, and J. G. Breitenbucher. 2005. Checkpoint kinase inhibitors: SAR and radioprotective properties of a series of 2-arylbenzimidazoles.
J. Med. Chem. 48:1873-1885.
Bernardi, R., P. P. Scaglioni, S. Bergmann, H. F. Horn, K. H. Vousden, and P. P. Pandolfi. 2004. PML regulates p53 stability by sequestering Mdm2 to the nucleolus. Nat. Cell Biol. 6:665-672.
Bradshaw, P. S., D. J. Stavropoulos, and M. S. Meyn. 2005. Human telomeric protein TRF2 associates with genomic double-strand breaks as an early response to DNA damage. Nat. Genet. 37:193-197.
Bristow, R. G. and R. P. Hill. 2005. Molecular & Cellular Basis of Radiotherapy. The Basic Science of Oncology, 4^{th} ed. Edited by Tannock, Hill, Bristow and Harrington, McGraw-Hill, New York. 261 pp.
Buscemi, G., C. Savio, L. Zannini, F. Micciche, D. Masnada, M. Nakanishi, H. Tauchi, K. Komatsu, S. Mizutani, K. Khanna, P Chen, P. Concannon, L. Chessa, and D. Delia 2001. Chk2 activation dependence on Nbs1 after DNA damage. Mol. Cell. Biol. 21:5214-5222.
Carbone, R, M. Pearson, S. Minucci, and P. G. Pelicci. 2002. PML NBs associate with the hMre11 complex and p53 at sites of irradiation induced DNA damage. Oncogene. 21:1633-1640.
Carney, J. P., R. S. Maser, H. Olivares, E. M. Davis, M. Le Beau, J. R. Yates 3rd, L. Hays, W. F. Morgan and J. H. Petrini. 1998. The hMre11/hRad50 protein complex and Nijmegen breakage syndrome: linkage of double-strand break repair to the cellular DNA damage response. Cell. 93:477-486.
Chubb, J. R., S. Boyle, P. Perry, and W. A. Bickmore. 2002. Chromatin motion is constrained by association with nuclear compartments in human cells. Curr. Biol. 12:439-445.
Compton, M. M. 1991. Development of an apoptosis endonuclease assay. DNA Cell Biol. 10(2):133-141.
D'Amours, D., and S. P. Jackson. 2002. The Mre11 complex: at the crossroads of dna repair and checkpoint signalling. Nat. Rev. Mol. Cell. Biol. 3:317-327.
Dellaire, G., R. Farrall, and W. A. Bickmore. 2003. The Nuclear Protein Database (NPD): subnuclear localisation and functional annotation of the nuclear proteome. Nucl. Acids Res. 31:328-330.
Dellaire, G., and D. P. Bazett-Jones. 2004. PML nuclear bodies: dynamic sensors of DNA damage and cellular stress. Bioessays. 26:963-977.
Dellaire, G., R. Nisman, and D. P. Bazett-Jones. 2004. Correlative light and electron spectroscopic imaging of chromatin in situ. Methods Enzymol. 375:456-478.
Dellaire, G., R. W. Ching, H. Dehghani, Y. Ren Y, and D. P. Bazett-Jones. 2006. The number of PML nuclear bodies increases in early S phase by a fission mechanism. J. Cell Sci. 119:1026-1033.
de Stanchina, E., E. Querido, M. Narita, R. V. Davuluri, P. P. Pandolfi, G. Ferbeyre, S. W. Lowe. 2004. PML is a direct p53 target that modulates p53 effector functions. Mol. Cell. 13:523-535.
D'Orazi, G., B. Cecchinelli, T. Bruno, I. Manni, Y. Higashimoto, S. Saito, M. Gostissa, S. Coen S, A. Marchetti, G. Del Sal, G. Piaggio G, M. Fanciulli, E. Appella, and S. Soddu. 2002. Homeodomain-interacting protein kinase-2 phosphorylates p53 at Ser 46 and mediates apoptosis. Nat. Cell Biol. 4:11-19.
Eskiw, C. H., G. Dellaire, J. S. Mymryk, and D. P. Bazett-Jones. 2003. Size, position and dynamic behavior of PML nuclear bodies following cell stress as a paradigm for supramolecular trafficking and assembly. J. Cell Sci. 116:4455-4466.
Eskiw, C. H., G. Dellaire, and D. P. Bazett-Jones. 2004. Chromatin contributes to structural integrity of promyelocytic leukemia bodies through a SUMO-1-independent mechanism. J. Biol. Chem. 279:9577-9585.
Everett, R. D. 2001. DNA viruses and viral proteins that interact with PML nuclear bodies. Oncogene. 20:7266-7273.
Everett, R. D., and J. Murray. 2005. ND10 components relocate to sites associated with herpes simplex virus type 1 nucleoprotein complexes during virus infection. J. Virol. 79:5078-5089.
Gerdes, J., U. Schwab, H. Lemke, and H. Stein. 1983. Production of a mouse monoclonal antibody reactive with a human nuclear antigen associated with cell proliferation. Int. J. Cancer. 31:13-20.
Gill, G. 2004. SUMO and ubiquitin in the nucleus: different functions, similar mechanisms? Genes Dev. 18:2046-2059.
Gorczyca, W., J. Gong, and Z. Darzynkiewicz. 1993 Detection of DNA strand breaks in individual apoptotic cells by the in situ terminal deoxynucleotidyl transferase and nick translation assays. Cancer Res. 53(8):1945-1951.
Gorisch, S. M., M. Wachsmuth, C. Ittrich, C. P. Bacher, K. Rippe, and P. Lichter 2004. Nuclear body movement is determined by chromatin accessibility and dynamics. Proc. Natl. Acad. Sci. USA. 101:13221-13226
Grobelny, J. V., A. K. Godwin, and D. Broccoli. 2000. ALT-associated PML bodies are present in viable cells and are enriched in cells in the G(2)/M phase of the cell cycle. J. Cell Sci. 113:4577-4585.
Guo, A., P. Salomoni, J. Luo, A. Shih, S. Zhong, W. Gu, and P. P. Pandolfi. 2000. The function of PML in p53-dependent apoptosis. Nat. Cell Biol. 2:730-736.
Hofmann, T. G., A. Moller, H. Sirma, H. Zentgraf, Y. Taya, W. Droge, H. Will, and M. L. Schmitz. 2002. Regulation of p53 activity by its interaction with homeodomain-interacting protein kinase-2. Nat. Cell Biol. 4:1-10.
Hu, P., S. F. Beresten, A. J. van Brabant, T. Z. Ye, P. P. Pandolfi, F. B. Johnson, L. Guarente and N. A. Ellis. 2001. Evidence for BLM and Topoisomerase IIIalpha interaction in genomic stability. Hum. Mol. Genet. 10:1287-1298.
Jensen, K., C. Shiels, and P. S. Freemont. PML protein isoforms and the RBCC/TRIM motif. Oncogene. 20(49):7223-7233.
Koi, M, A. Umar, D. P. Chauhan, S. P. Cheman, J. M. Carethers, A. Kunkel, and C. R. Boland. 1994. Human chromosome 3 corrects mismatch repair deficiency and microsatellite instability and reduces N-methyl-N′-nitro-N-nitrosoguanidine tolerance in colon tumor cells with homozygous hMLH1 mutation. Cancer Res. 54:4308-4312
Kruhlak, M. J., A. Celeste, G. Dellaire, O. Fernandez-Capetillo, W. G. Muller, J. G. McNally, D. P. Bazett-Jones D P, and A. Nussenzweig. 2006. Changes in chromatin structure and mobility in living cells at sites of DNA double-strand breaks. J. Cell Biol. 2006 172:823-834.
Kurz, E. U., and S. P. Lees-Miller. 2004. DNA damage-induced activation of ATM and ATM-dependent signaling pathways. DNA Repair 3:889-900.
Lin, H. K., S. Bergmann, P. P Pandolfi. Deregulated TGF-beta signaling in leukemogenesis. Oncogene. 24(37):5693-5700.
Lydall D, and S. Whitehall. 2005 Chromatin and the DNA damage response.
DNA Repair 4:1195-1207.
Nghiem, P., P. K. Park, Y. Kim, C. Vaziri, and S. L. Schreiber. 2001. ATR inhibition selectively sensitizes G1 checkpoint-deficient cells to lethal premature chromatin condensation. Proc. Natl. Acad. Sci. USA 98:9092-9097.
O'Driscoll, M., V. L. Ruiz-Perez, C. G. Woods, P. A. Jeggo, and J. A. Goodship. 2003. A splicing mutation affecting expression of ataxia-telangiectasia and Rad3-related protein (ATR) results in Seckel syndrome. Nat. Genet. 33:497-501.
Olive, P. L., J. P. Banath, and R. E. Durand. 1990. Heterogeneity in radiation-induced DNA damage and repair in tumor and normal cells measured using the “comet” assay. Radiat Res. 122:86-94.
Pommier, Y., O, Sordet, V. A. Rao, H. Zhang, and K. W. Kohn. 2005. Targeting chk2 kinase: molecular interaction maps and therapeutic rationale. Curr. Pharm. Des. 11:2855-2872.
Rao, V. A., A. M. Fan, L. Meng, C. F. Doe, P. S. North, I. D. Hickson, and Y. Pommier. 2005. Phosphorylation of BLM, dissociation from topoisomerase IIIalpha, and colocalization with gamma-H2AX after topoisomerase I-induced replication damage. Mol. Cell Biol. 25:8925-8937.
Rogakou, E. P., C. Boon, C. Redon, and W. M. Bonner. 1999. Megabase chromatin domains involved in DNA double-strand breaks in vivo. J. Cell Biol. 146:905-916.
Salomoni, P., and P. P. Pandolfi P P. 2002. The role of PML in tumor suppression. Cell. 108:165-170.
Seker, H., C. Rubbi, S. P. Linke, E. D. Bowman, S. Garfield, L. Hansen, K. L. Borden, J. Milner and C. C. Harris. 2003. UV-C-induced DNA damage leads to p53-dependent nuclear trafficking of PML. Oncogene. 22:1620-1628.
Shiloh, Y. 2001. ATM and ATR: networking cellular responses to DNA damage. Curr. Opin. Genet. Dev. 11:71-77.
Stewart, G. S., R. S. Maser, T. Stankovic, D. A. Bressan, M. I. Kaplan, N. G. Jaspers, A. Raams, P. J. Byrd, J. H. Petrini, and A. M. Taylor. 1999. The DNA double-strand break repair gene hMRE11 is mutated in individuals with an ataxia-telangiectasia-like disorder. Cell. 99:577-587.
Stracker, T. H., J. W. Theunissen, M. Morales, J. H. Petrini. 2004 The Mre11 complex and the metabolism of chromosome breaks: the importance of communicating and holding things together. DNA Repair 3(8-9):845-854.
Trotman, L. C., A. Alimonti, P. P Scaglioni, J. A. Koutcher, C. Cordon-Cardo, P. P Pandolfi. Identification of a tumour suppressor network opposing nuclear Akt function. Nature. 441(7092):523-527.
Xu, Z. X., A. Timanova-Atanasova, R. X. Zhao, and K. S. Chang K S. 2003. PML colocalizes with and stabilizes the DNA damage response protein TopBP1.
Mol. Cell. Biol. 23:4247-4256.
Yang, S., C. Kuo, J. E. Bisi, and M. K. Kim. 2002. PML-dependent apoptosis after DNA damage is regulated by the checkpoint kinase hCds1/Chk2.
Nat. Cell Biol. 4:865-870.
Yeager, T. R., A. A. Neumann, A. Englezou, L. I. Huschtscha, J. R. Noble, and R. R. Reddel. 1999. Telomerase-negative immortalized human cells contain a novel type of promyelocytic leukemia (PML) body. Cancer Res. 59:4175-4179.
Zhong, S., P. Salomoni, and P. P. Pandolfi P P. 2000. The transcriptional role of PML and the nuclear body. Nat Cell Biol. 2:E85-90.

1. A method for assessing or monitoring efficacy of a DNA-damaging agent in the treatment of a tumor in a subject, the method comprising:
contacting tumor tissue with the DNA-damaging agent;
determining the value of a selected promyelocytic leukemia nuclear body (PML NB) property in the tumor tissue at one or more time points after the contact with the DNA-damaging agent; and
comparing the value of the selected property in the tumor tissue with the value of the selected property in control tissue;
wherein a difference between the value of the selected property in the tumor tissue and the value of the property in the control tissue indicates efficacy for treatment of the tumor.

2. A method for assessing or monitoring efficacy of a DNA-damaging agent in the treatment of a tumor in a subject, the method comprising:
contacting tumor tissue and normal tissue with the DNA-damaging agent; and
determining the value of a selected PML NB property in the tumor tissue and in the normal tissue at one or more time points after the contact with the DNA-damaging agent
wherein a difference in the response or in the timing of the response to the agent between the tumor and the normal tissue indicates that the agent lacks efficacy for treatment of the tumor.

3-18. (canceled)

19. The method of claim 2, wherein the normal tissue and the tumor tissue are obtained from a single biopsy sample from the subject.

20. The method of claim 2, wherein the normal tissue is normal tissue corresponding to the tumor tissue.

21. (canceled)

22. The method of claim 1 or 2, wherein the PML NB property is selected from the group consisting of:
a PML NB characteristic that changes in response to DNA damage;
PML NB number;
PML NB biochemical composition;
PML NB morphology;
PML NB behaviour;
a profile of PML NB number at selected time intervals;
a profile of PML NB composition at selected time intervals;
a profile of PML NB morphology at selected time intervals; and
a profile of PML NB behavior at selected time intervals.

23. The method of claim 22 wherein the PML NB behaviour is motility.

24. The method of claim 22 wherein the PML NB property is PML NB number.

25. The method of claim 22, wherein the DNA-damaging agent is radiation, a chemotherapeutic compound, or a chemical that generates reactive oxygen species.

26. The method of claim 25 wherein the radiation is ionizing radiation, gamma-radiation, alpha particle radiation or ultra violet radiation.

27. The method of claim 26 wherein the ionizing radiation is X radiation.

28. (canceled)

29. The method of claim 25 wherein the chemotherapeutic compound is a reactive oxygen species, a topoisomerase I inhibitor, a topoisomerase II inhibitor, a DNA alkylating/cross-linking agent, a bioflavonoid or a radiomimetic.

30. (canceled)

31. The method of claim 25 wherein the chemical is at least one of hydrogen peroxide, sodium chromate or sodium arsenite.

32. (canceled)

33. The method of claim 22, wherein the subject is a human subject.

34. The method of claim 1, wherein the control tissue is a sample of tumor tissue not contacted with the DNA-damaging agent and wherein the method is for assessing potential efficacy of the DNA-damaging agent.

35. The method of claim 1, wherein the control tissue is a sample of the tumor that was obtained prior to administration of the DNA-damaging agent and the method is for monitoring efficacy of the DNA-damaging agent.

36. The method of claim 2, further comprising:
comparing the value of the selected property in the tumor tissue and in the normal tissue with the respective values of the selected property in a control tumor tissue and a control normal tissue not contacted with the DNA-damaging agent to provide an indication of the response of the tumor tissue and the normal tissue to the agent;
wherein a difference in the response to the agent between the tumor and the normal tissue indicates that the agent lacks efficacy for treatment of the tumor.

37. The method of claim 36, wherein the value of a selected PML NB property in the tumor tissue and in the normal tissue are determined at a plurality of time points after the contact with the DNA-damaging agent;
wherein a difference in the timing of the response to the agent between the tumor and the normal tissue indicates that the agent lacks efficacy for treatment of the tumor.

38. A method for monitoring the response of a cell or tissue to stress, the method comprising determining the level of at least one PML NB property in the cell or tissue before and after the stress, or comparing the post-stress level of at least one PML NB property in the cell or tissue to the level in a corresponding normal unstressed cell or tissue, wherein the greater the difference in the level of the at least one PML NB property in the stressed cell or tissue compared with the level in the cell or tissue before the stress, or with the level in a corresponding normal unstressed cell or tissue, the greater the response of the cell or tissue to the stress.

39. The method of claim 38 wherein the stress is due to at least one of a DNA-damaging agent, hypoxia, anoxia, ischemia, oxygen deprivation, heat or a viral infection.

40. A method for monitoring oncogenesis and/or for assessing malignancy in a tissue in a subject, the method comprising determining the level of at least one PML NB property in the tissue, and comparing the level in the tissue to the level of the PML NB property in a control tissue, wherein a difference in the level of the property in the tissue of the subject relative to the level in the control tissue suggests increased oncogenesis and/or malignancy in the tissue of the subject.

41. The method of claim 40, wherein the tissue is tumor tissue and the control tissue is normal tissue and wherein the greater the difference in the level of the property in the tumor tissue relative to the level in the normal tissue, the greater the likely malignancy of the tumor.

42. The method of claim 1, wherein the selected PML NB property is PML NB number and wherein a lack of increase in PML NB number in the tumor tissue at one or more time points after contact with the DNA-damaging agent compared to the PML NB number of the control tissue indicates that the agent lacks efficacy for treatment of the tumor.

43. The method of claim 2 wherein the selected PML NB property is PML NB number and wherein a difference in the PML NB number response between the tumor and the normal tissue indicates that the agent lacks efficacy for treatment of the tumor.

44. The method of claim 2 wherein the selected PML NB property is PML NB number and wherein a difference in the timing of the PML NB number response between the tumor and the normal tissue indicates that the agent lacks efficacy for treatment of the tumor.

45. The method of claim 44 wherein a delayed increase in PML NB number in the tumor relative to the timing of a PML NB increase observed in the normal tissue indicates that the agent lacks efficacy for treatment of the tumor.
